SEQUENCE LISTING (20 sequences)

SEQ ID NO: 1 - DNA, 22 bp, Artificial Sequence
  Forward Primer for HSPA1A gene
caggtgatcaacgacggagaca

SEQ ID NO: 2 - DNA, 22 bp, Artificial Sequence
  Reverse Primer for HSPA1A gene
gtcgatcgtcaggatggacacg

SEQ ID NO: 3 - DNA, 19 bp, Artificial Sequence
  Forward Primer for HSPA6 gene
ccgtgaagcacgcagtgat

SEQ ID NO: 4 - DNA, 19 bp, Artificial Sequence
  Reverse Primer for HSPA6 gene
acgagccggttgtcgaagt

SEQ ID NO: 5 - DNA, 20 bp, Artificial Sequence
  Forward Primer for RPM3 gene
cttcagcagtttcggaccta

SEQ ID NO: 6 - DNA, 20 bp, Artificial Sequence
  Reverse Primer for RBM3 gene
accatccagagactctccgt

SEQ ID NO: 7 - DNA, 22 bp, Artificial Sequence
  Forward Primer for CIRBP gene
caaagtacggacagatctctga

SEQ ID NO: 8 - DNA, 19 bp, Artificial Sequence
  Reverse Primer for CIRBP gene
cggatctgccgtccatcta

SEQ ID NO: 9 - DNA, 22 bp, Artificial Sequence
  Forward Primer for TATABP gene
gaatataatcccaagcggtttg

SEQ ID NO: 10 - DNA, 20 bp, Artificial Sequence
  Reverse Primer for TATABP gene
acttcacatcacagctcccc

SEQ ID NO: 11 - RNA, 19 nt, Artificial Sequence
  siRNA for CIRBP
gguccuacagagacaguua

SEQ ID NO: 12 - RNA, 19 nt, Artificial Sequence
  siRNA for CIRBP
gaacauugacgacgcuaag

SEQ ID NO: 13 - RNA, 19 nt, Artificial Sequence
  siRNA for CIRBP
gagcaggucuucucaaagu

SEQ ID NO: 14 - RNA, 19 nt, Artificial Sequence
  siRNA for CIRBP
ccagagaucucggggauuu

SEQ ID NO: 15 - RNA, 19 nt, Artificial Sequence
  siRNA for RBM3
acaugauuauccagcggua

SEQ ID NO: 16 - RNA, 19 nt, Artificial Sequence
  siRNA for RBM3
agacaaauuaugggacguu

SEQ ID NO: 17 - RNA, 19 nt, Artificial Sequence
  siRNA for RBM3
gugggagggcucaacuuua

SEQ ID NO: 18 - RNA, 19 nt, Artificial Sequence
  siRNA for RBM3
ggaucaugcaggcaagucu

SEQ ID NO: 19 - protein, 23 aa, Artificial Sequence
  RNP-1 RNA-recognition motif
AVFSLSQPEQVKIAVNTSKYAES

SEQ ID NO: 20 - protein, 25 aa, Artificial Sequence
  RNP-2 RNA-recognition motif
VLHVTFPKEWKTSDLYQLFSAFGNI

(19) United States
(12) Patent Application Publication
(10) Pub. No.: US 2011/0313233 A1
(43) Pub. Date: Dec. 22, 2011

(54) DOWN-REGULATION OF COLD SHOCK PROTEINS FOR CANCER TREATMENT

(75) Inventors: Robert H. Getzenberg, Pittsburgh, PA (US); Prakash Kulkarni, Clarksville, MD (US); Yu Zeng, Baltimore, MD (US)

(73) Assignee: THE JOHNS HOPKINS UNIVERSITY, Baltimore, MD (US)

(21) Appl. No.: 12/817,448

(22) Filed: Jun. 17, 2010

(51) Int. Cl. A61M 37/00; A61P 35/00; A61N 5/00; C12Q 1/68; A61N 2/00; A61K 31/713; C12Q 1/02

(52) U.S. Cl. 600/3; 514/44 A; 435/29; 435/6.1; 604/20; 600/9

(57) ABSTRACT

The present invention is in the field of treatment of diseased tissues, including cancerous tissues. In one embodiment, the present invention provides methods of identifying tissues that down-regulate cold shock proteins in response to environmental stresses, such as heat. The present invention also provides methods of treatment of diseased tissues comprising down-regulation of cold shock proteins, as well as environmentally stressing (e.g., heating) the tissues, in combination with one or more additional therapies.

### BACKGROUND OF THE INVENTION

1. Field of the Invention

The present invention is in the field of treatment of diseased tissues, including cancerous tissues. In one embodiment, the present invention provides methods of identifying tissues that down-regulate cold shock proteins in response to environmental stresses, such as heat. The present invention also provides methods of treatment of diseased tissues comprising down-regulation of cold shock proteins, as well as environmentally stressing (e.g., heating) the tissues, in combination with one or more additional therapies.

2. Background Art

Despite decades of intense research efforts worldwide, cancer remains a major healthcare concern and is the second leading cause of death in the western world. According to recent estimates by the American Cancer Society, cancer claims more than 500,000 lives each year in the United States alone. Traditional treatments are either invasive or expose the patient to considerable side effects with often only modest positive outcomes. Better diagnostic practices and advancements in technology have improved early detection and prognosis for many patients but many types of cancers defy current treatment options despite these improvements. Of the many forms of cancer that still pose a medical challenge, prostate, breast, lung, and liver claim the vast majority of lives each year.

Recent therapeutic advances allow most cancer patients to achieve clinical responses. However, while clinical responses can clearly decrease side effects and improve quality of life, most cancer patients still relapse and the of their disease. Emerging data suggest that initial responses in cancer represent therapeutic effectiveness against the differentiated cancer cells making up the hulk of the tumor. However, the rare biologically distinct cancer stem cells resistant to current therapies are responsible for relapse. The age of the patient, extent of distant metastasis, especially micrometastasis, and response to initial therapy are important in predicting prognosis. Since prostate cancer affects older men, many die of other causes before the advancing disease causes discernable symptoms making treatment selection difficult.

Metastasis is a complex multi-step process that results in spread of tumorigenic cells to secondary sites in various organs. Upon growth of neoplastic cells beyond a certain mass (2 mm in diameter) an extensive vascularization through angiogenesis occurs. Through angiogenesis, vascular endothelial cells provide the supply of nutrients for the growth of the primary tumor mass and the route of extravasation. Thus, treatments that also target endothelial cells and cancer stem cells are more likely to increase patient response and have the potential to revolutionize the treatment of many cancers.

It is hypothesized that testicular cancer patients have a higher survival rate than other cancer patients because the cancer cells are sensitive to body beat, a concept termed the “Lance Armstrong Effect” (3). The Lance Armstrong effect might primarily result from the unusual thermal sensitivity of normal testicular germ cells and their propensity to die when placed at the normal body temperature of 37° C. The metastatic testicular cancer cells that spread may retain this hyperthermic stress response to body temperature that would enhance their destruction through increased sensitivity to therapeutic-induced cell death due to radiation or chemotherapy. Numerous clinical and basic studies have shown that hyperthermic stress can alter tumor cell kill and survival in a significant manner both in vivo and in vitro. In addition, in many tumor types, hyperthermia (suitably 41° C. to 43° C.) increases and synergizes the therapeutic response to combination therapy, such as radiation, cytotoxic drugs and immunotherapy. Hyperthermia has been used alone and in combination with other forms of cancer therapy fix many years but with only marginal clinical success. More recently, there have been a few clinical trials showing significant benefit of the addition of heat to radiation and chemotherapy. However, variability is often observed between cell types making generalizations of heat effects difficult.

There is therefore a need for improved therapeutic modalities to treat various cancers. Furthermore, it would be highly desirable for such therapies to be minimally invasive and to target diseased tissues while sparing the unaffected healthy ones and preferably, to be administered to the patient in a typical medical facility setting.

### BRIEF SUMMARY OF THE INVENTION

The present invention fulfills the needs identified above by providing methods of treatment of diseased tissues (including cancerous tissues) in patients, for example, by heating. In addition, the present invention provides additional methods of treatment of diseased tissues by down-regulating cold shock proteins in diseased tissues.

In one embodiment, the present invention provides methods of treating a patient suffering from a diseased tissue. Suitably, such methods comprise administering to the diseased tissue of the patient, one or more nucleic acid molecules that down-regulate one or more cold shock proteins in the diseased tissue. The susceptibility of the diseased tissue to an additional therapy is enhanced relative to diseased tissue which has not been administered one or more nucleic acids. As described herein, suitably, the nucleic acid molecules are siRNA, including siRNA that down-regulate the cold shock proteins RBM3 and/or CIRBP. In exemplary embodiments, the diseased tissue is a cancerous tissue, such as a cancerous tissue of the heart, lung, breast, prostate, bladder, pancreas, brain, stomach, kidney, testes, lymph nodes, skin, bone or bone marrow.

In embodiments, the methods further comprise administering to the patient an additional therapy, including administering a chemotherapeutic agent, radiation therapy, immunotherapy, radio-immunotherapy, gene therapy and gene silencing therapy. Exemplary chemotherapeutic agents that can he administered, include, but are not limited to methotrexate, adriamycin, epirubicin, daunorubicin, doxorubicin, amphotericin B, vincristine, vinblastine, etoposide, ellipticine, camptothecin, paclitaxel, docetaxol, cisplatin, prednisone, methyl-prednisone, and navalbene. In further embodiments, the methods further comprise administering one or more additional nucleic acid molecules to the diseased tissue that down-regulate one or more heat shock proteins in the diseased tissue. Suitably, the additional nucleic acid molecules are siRNA.

The methods of the present invention are suitably used to treat mammalian patients, including humans.

In further embodiments, the present invention provides additional methods of treating a patient suffering from a diseased tissue. Such methods suitably comprise environmentally stressing the diseased tissue of the patient for a period of greater than about 10 minutes. One or more nucleic acid molecules that down-regulate one or more cold shock proteins in the diseased tissue are administering to the diseased tissue of the patient. An additional therapy is administered to the patient. Suitably, such additional therapy is administration of a chemotherapeutic agent, radiation therapy, immunotherapy, radio-immunotherapy, gene therapy and/or gene silencing therapy. The susceptibility of the diseased tissue to the additional therapy is enhanced relative to diseased tissue which has not been environmentally stressed and administered one or more nucleic acids.

In exemplary embodiments, the environmental stressing comprises locally heating the diseased tissue, so as to raise the temperature of the diseased tissue to about 39° C. to about 41° C. for a period of greater than about 10 minutes. As noted herein, suitably the tissue is a cancerous tissue. In suitable embodiments, the temperature is raised for a period of about 30 minutes to about 24 hours, for example, for a period of about 4 hours to about 8 hours. Suitably, the temperature is raised to about 39° C., to about 40° C., or to about 41° C.

In exemplary embodiments, the local heating comprises application of non-ionizing electromagnetic radiation, ionizing radiation Or sound energy to the diseased tissue. In further embodiments, the local heating comprises administering a magnetic material to the patient and applying an alternating magnetic field so as to inductively heat the magnetic material.

The present invention also provides methods of identifying a tissue in which cold shock proteins are down-regulated in response to environmental stressing of the tissue. Such methods suitably comprise environmentally stressing the tissue for a period of greater than about 10 minutes, and assaying the tissue for expression of one or more cold shock proteins. The expression of the cold shock proteins are compared to the expression of cold shock proteins in a sample of the tissue that has not been environmentally stressed, wherein a decrease in the expression in the environmentally stressed sample relative to the expression in the non-environmentally stressed sample identifies the environmentally stressed sample as a tissue in which cold shock proteins are down-regulated in response to the environmental stressing. As described herein, suitably the environmental stressing comprises heating the tissue, so as to raise the temperature of the tissue to about 39° C. to about 41° C. for a period of greater than about 10 minutes.

The assaying for expression suitably comprises analysis of RNA from the tissue, or can comprise analysis of protein from the tissue. Suitably, the tissue is a mammalian tissue, such as a human tissue.

In still further embodiments, the present invention provides methods of treating a patient suffering from a diseased tissue. Such methods comprise identifying a diseased tissue of the patient in which cold shock proteins are down-regulated in response to environmental stressing of the diseased tissue. The diseased tissue of the patient is environmentally stressed for a period of greater than about 10 minutes, wherein the susceptibility of the diseased tissue to an additional therapy is enhanced relative to diseased tissue that has riot been environmentally stressed. As described herein, suitably, the environmental stressing comprises locally heating the diseased tissue, so as to raise the temperature of the tissue to about 39° C. to about 41° C. for a period of greater than about 10 minutes. In exemplary embodiments, one or more additional therapies are administered to the patient. As described herein, such therapies can include administration of a chemotherapeutic agent, radiation therapy, immunotherapy, radio-immunotherapy, gene therapy and gene silencing therapy. The methods can further comprise administering one or more nucleic acid molecules to the tissue that down-regulate one or more heat shock proteins in the tissue.

### BRIEF DESCRIPTION OF THE DRAWINGS/FIGURES

FIG. 1 shows an exemplary experimental design for mapping cellular response to heat treatment.

FIGS. 2A-2H show representative gene expression changes in a microarray analysis in two prostate cancer cell lines.

FIGS. 3A-3B show a western blot showing levels of Hspa1a, RBM3 and CIRBP proteins in two prostate cancer cell lines.

FIG. 4 shows the increase in heating as a function of magnetic field strength for magnetic nanoparticles.

FIGS. 5A-5C show the targeting of magnetic nanoparticles to cancer cells.

FIG. 6 shows MALDI-TOF and peptide mass fingerprinting analysis of heat shock response nuclear matrix protein.

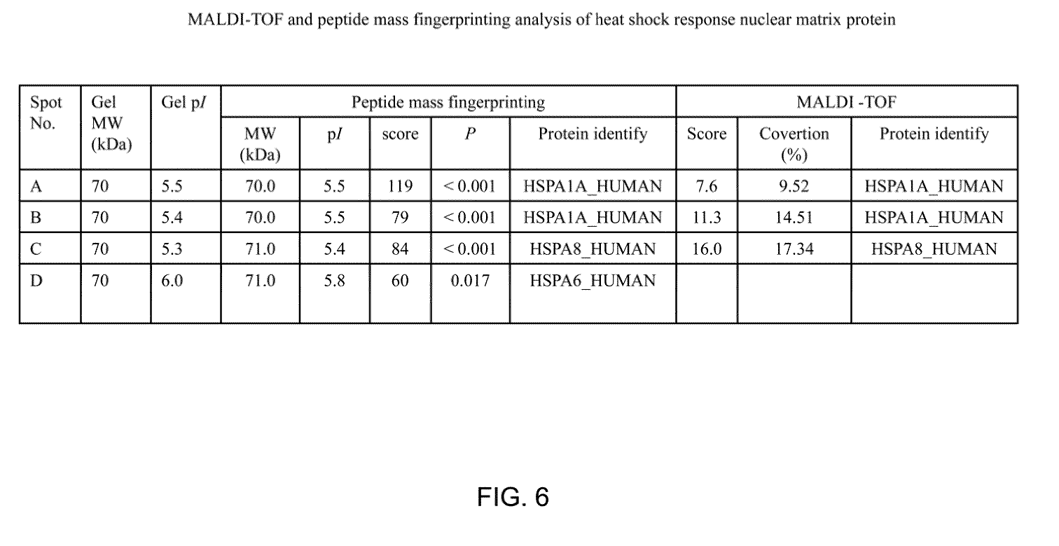

FIGS. 7A-7D show mapping of nuclear matrix proteins (NMPs) changes in response to heat in prostate cancer cells PC-3 and LNCaP.

FIGS. 8A-8D show the effects of knocking down RBM3 and CIRBP by siRNA on chemosensitivity in PC-3 cells and LNCaP cells.

FIGS. 9A and 9B show the enhancement of RBM3 and CIRBP knockdown on chemosensitivity confirmed by detecting apoptosis using the Terminal Transferase dUTP Nick End Labeling (TUNEL) assay.

FIGS. 10A-10D demonstrate the synergistic effects of mild heat treatment and chemotherapy in prostate cancer cells showing cell viability of PC-3 cells and LNCaP cells treated at 41° C. for 4 h, 24 h, or maintained at 37° C. as a control, cDDP or ADM were added during the heat treatment and washed out 4 h later.

FIGS. 11A and 11B show the effects of down regulation of RBM3 and CIRBP on the cell cycle in LNCaP (11A) and PC-3 (11B) cells transfected with 100 nM RBM3, CIRBP siRNA or scrambled control (si-Scrambled).

FIGS. 12A and 12B show western blots representing the effects of RBM3 and CIRBP down-regulation in prostate cancer cells PC-3 (A) and LNCaP (B) on the levels of Cyclin B1, Cyclin D1, phosph-histone H2A.X (Ser139) (γ-H2A.X), phosph-p53 (Ser15), and p21. Actin was used as a control for protein loading.

FIGS. 13A-13H: Show the effects of down-regulation of RBM3 and CIRBP on colony formation in prostate cancer cells, PC-3 and LNCaP.

### DETAILED DESCRIPTION OF THE INVENTION

Heat is a critical microenvironmental factor for regulating imprinting, differentiation and replication in biological systems. Imprinting, differentiation and replication are all central issues in the control of cancer and in enhancing radiation, chemotherapy and immunotherapy effects not only in cancer cells, but also in endothelial cells and cancer stem cells as well. Excessive heat (40° C.-46° C.) relative to normal human body temperature (37° C.), at short durations (minutes to hours), can cause irreversible damage to tumor cells without affecting normal cells. It should he understood that “normal” body temperature can be higher or lower than 37° C. depending on the type of mammal. However, from a therapeutic perspective, the tumor cell's defense mechanisms specifically, the heat shock proteins, pose a severe impediment. While exposing tumor tissue to higher temperatures or extending the duration of heat treatments may overcome cellular defense mechanisms, this has been difficult to accomplish in clinical practice. Thus, understanding the molecular mechanisms that underlie the increased sensitivity of cancer cells to heat, and the synergism with commonly used therapeutic approaches, provides insights to enhance cures of solid tumors that remain refractory to current systemic treatments.

The most sensitive cellular target of heat is the nuclear matrix, a dynamic scaffold that organizes many functions within the nucleus. It plays an important role in several cellular activities such as maintaining cell morphology, three-dimensional organization of the nucleus, DNA replication and transcription. As a result, the nuclear matrix is intimately associated with cell proliferation and differentiation, as well as with carcinogenesis (4, 5). In cancer cells, the nuclear matrix is not only abnormal in morphology, but is also different in its composition (6). Studies of the thermal effects on the nucleus by Roti-Roti et al. (7) and Lopock et al. (8) have demonstrated thermally induced unfolding of the nuclear matrix and subsequent changes in the binding of specific proteins to the matrix. Taken together these data strongly suggest that changes in the structure and composition of the nuclear matrix in response to heat treatment of cancer cells may account for some of the underlying mechanisms. However, despite significant progress in uncovering the effects of heat shock on cellular processes (9), understanding the molecular mechanisms underlying its synergistic effects in treating cancer still represent significant challenges. Thus, in one embodiment, as described herein, the present invention utilizes new pathways that can be pharmacologically manipulated to synergize with traditional therapeutic methods, such as radiation and chemotherapy, as a therapeutic approach for treatment of diseased tissues including cancer.

The present invention is based in part on the discovery that local, mild hyperthermic temperatures down-regulate the expression of cold shock proteins in tissues. This down-regulation also results in enhanced susceptibility of the tissues to additional therapeutic treatments.

In one embodiment, the present invention provides methods of identifying as tissue in which cold shock proteins are down-regulated in response to treatment of the tissue with one or more environmental stresses, such as heat. The methods suitably comprise subjecting as patient's diseased tissue to an environmental stress for a period of greater than about 10 minutes. Suitably, such methods comprise heating the tissue, so as to raise the temperature of the tissue to mild hyperthermic temperatures, suitably to about 39° C. to about 41° C., for a period of greater than about 10 minutes. The tissue is then assayed for expression of one or more cold shock proteins. The expression of the cold shock proteins is compared to the expression of cold shock proteins in a sample of the tissue that has not been stressed, e.g., heated. A decrease in the expression in the stressed (e.g., heated) sample of tissue, relative to expression in the non-stressed sample, identifies the stressed sample as a tissue in which cold shock proteins are down-regulated in response to the stress.

Environmental stresses, in addition to heat, to which the tissues can be subjected include, but are not limited to, one or more of cold, pressure (increased or decreased relative to atmospheric pressure), pH (increased or decreased relative to physiologic pH (˜7.4)), light (increased or decreased relative to ambient conditions for the cell, tissue, organ or organism), sound (increased or decreased relative to ambient conditions for the cell, tissue, organ or organism), gravity (increased or decreased relative to ambient conditions for the cell, tissue, organ or organism) etc. As used herein, “environmentally stressing” a tissue of a patient refers to subjecting or exposing the tissue of the patient to the specified environmental stress(es).

In exemplary embodiments where the environmental stress is heating, the temperature of the diseased tissue is raised to the stressing temperature for a period of about 10 minutes to about 24 hours, suitably about 30 minutes to about 24 hours, about 1 hour to about 12 hours, about 4 hours to about 8 hours, or about 1 hour, about 2 hours, about 3 hours, about 4 hours, about 5 hours, about 6 hours, about 7 hours, about 8 hours, about 9 hours, about 10 hours, about 11 hours or about 12 hours. As used herein, the term “for a period” is used to indicate that the environmental stress is maintained for at least the recited length of time. For example, the temperature of the diseased tissue is maintained at a temperature within the recited range (or at the specific temperature) for the duration of the recited period. It should be understood that the temperature can vary within the recited range, or near the recited temperature, during, the heating period, and still be considered to be raised for that period of time. The term “about” as used herein refers to a range of ±10% of a value. For example, “about 10 minutes” refers to a range of time of 9 minutes to 11 minutes, inclusive.

The term “mild hyperthermia” is used herein to refer to the heating of a tissue to a temperature of about 39° C. to about 41° C., and any temperature or range of temperatures between 39° C. to about 41° C. Suitably, the heating is to a temperature of about 39° C. to about 40° C., about 40° C. to about 41° C., or about 39° C., about 39.5° C.,about 40° C., about 40.5° C., or about 41° C. For example, “about 40° C.” refers to a range of temperatures of 39.6° C. to 40.4° C., inclusive.

As used herein, the term “down-regulated” when referring to the down-regulation of proteins, means that the expression of the protein in a cell is reduced or eliminated in response to a stimulus (e.g., heat or other environmental stress, or via an interaction with nucleic acid molecules, such as siRNA or antisense), relative to the expression of the same protein in a cell that has not been exposed to the stimulus. Expression of proteins includes the amount of mRNA that is produced and/or the amount of ultimate protein that is produced by a cell.

Methods of assaying for the expression of proteins are well known in the art and include analysis of the amount of RNA from the tissues (such as mRNA). For example, gel electrophoresis and Northern blots can be utilized, as described in “Current Protocols in Molecular Biology,” Chapter 4, Ausubel et al., Eds., John Wiley & Sons, Inc., New York (1997), the disclosure of which is incorporated by reference herein m its entirety. The expression of proteins can also be measured by analyzing the amount of protein directly, for example, by gel electrophoresis and Western blots, spectrophotometric methods, immunoblotting, chromatographic methods, etc., as disclosed in “Current Protocols in Molecular Biology,” Chapter 10, incorporated by reference herein in its entirety. In addition, down-regulation of proteins can also be analyzed via various flow cytometry methods, e.g., utilizing fluorescent labeling of surface proteins. Additional methods of RNA and/or protein analysis are well known by those of ordinary skill in the art. Analyzing the expression of a protein can include measurement of the amount of RNA and/or protein utilizing a quantitative or qualitative method to determine if the expression has been reduced.

As used herein the term “tissue” includes single cells as well as aggregates (i.e., two or more) of cells of any type. Suitably, the tissue is a mammalian tissue, such as tissue from a dog, cat, horse, pig, mouse, rat, goat or primate (e.g., human), although tissues from fish and birds can also be assayed and/or treated using the various methods described herein. The tissue can be a normal tissue or a diseased tissue, such as a cancerous tissue. As used herein, “cancerous tissue” includes solid tumors, as well as metastatic and non-solid tumors. The tissue can be from any organ or part of the body, including for example, heart, lung, breast, prostate, bladder, pancreas, brain, stomach, kidney, testes, lymph node, skin, bone, bone marrow, etc.

As described herein, exemplary methods of the present invention can be utilized to identify a tissue in which cold shock proteins are down-regulated in response to an environmental stress, such as heating of the tissue. Cold shock proteins are a group of proteins expressed as a result of the reduction of the temperature of a cell below normal physiological temperature (i.e., about 37° C. for humans). Exemplary cold shock proteins that have been identified include RNA binding motif protein 3 (RBM3), cold inducible RNA binding protein (CIRBP), etc. See, e.g., Al-Fageeh M B & Smales C M “Control and regulation of the cellular responses to cold shock: the responses in yeast and mammalian systems,” Biochem. J. 397:247-259 (2006).

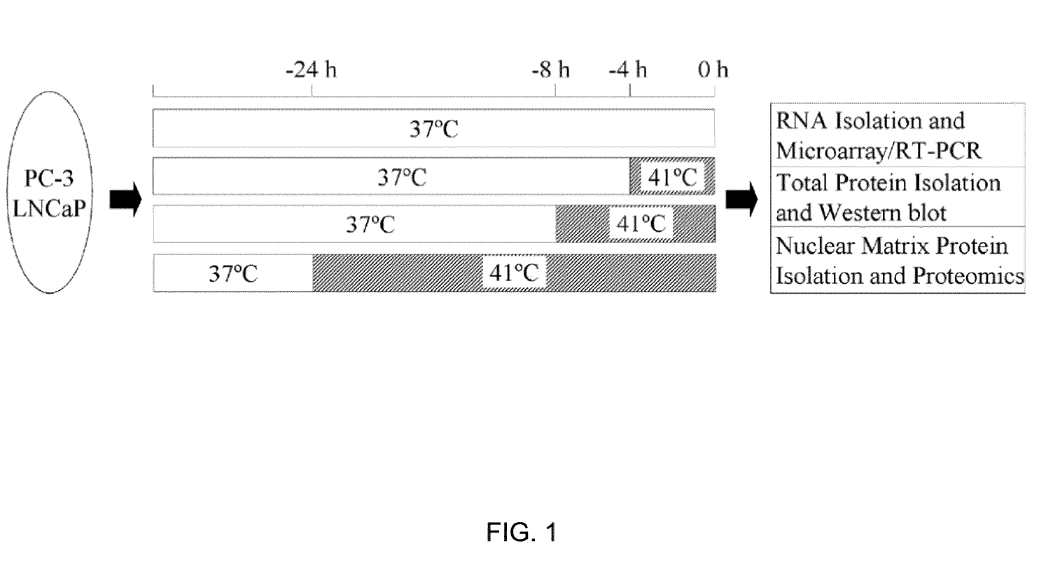

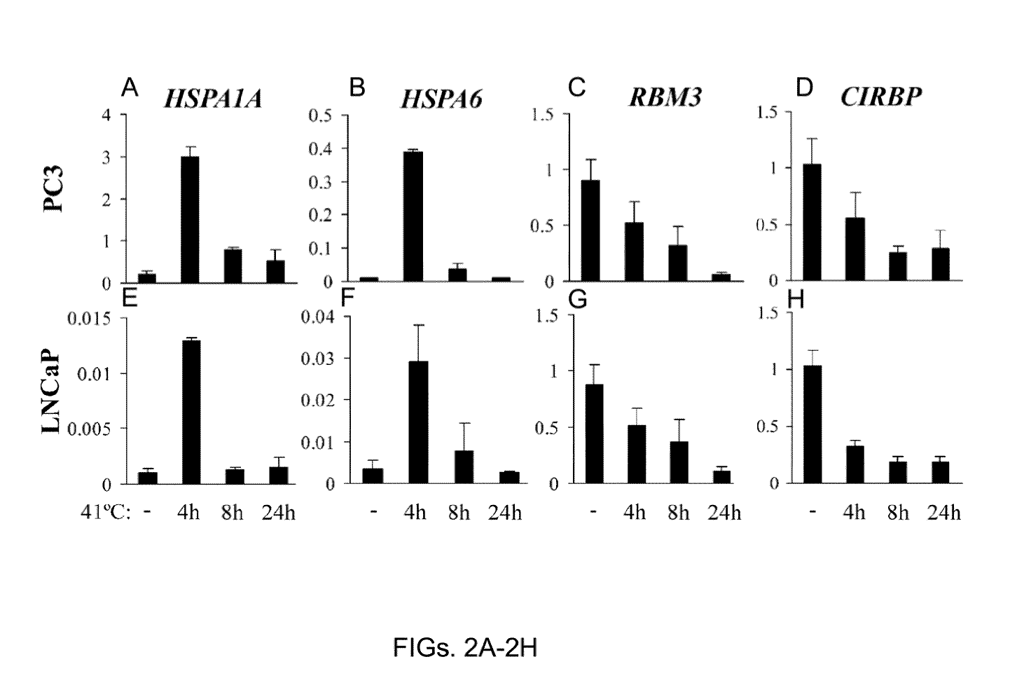

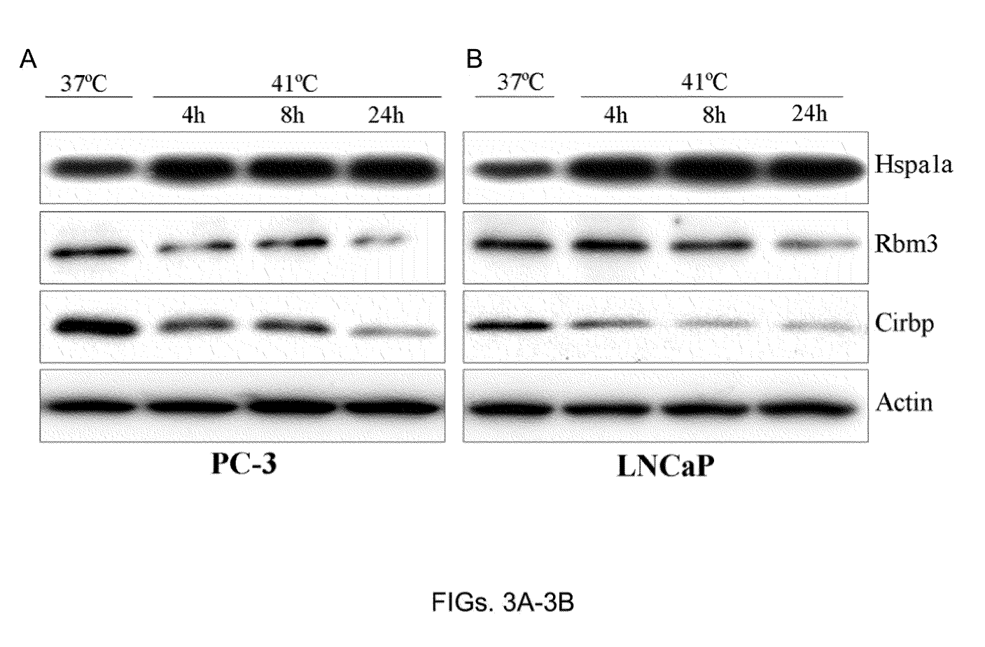

A suitable experimental design for mapping cellular response to heat treatment is shown in FIG. 1. Cells (such as prostate cancer cells PC-3 and LNCaP) were cultured at a suitable temperature, e.g., 41° C., for a pre-determined duration and subjected to RNA and/or protein analysis. As shown in FIGS. 2A-2H, heating of prostate cancer cell lines LNCaP and PC-3 to 41° C. for 4, 8 and 24 hours resulted in an increase in the expression of the heat shock proteins HSPAIA and HSPA6 relative to the expression in unheated cells (FIGS. 2A, 2B, 2E and 2F). However, the cold shock proteins RBM3 and CIRBP were down regulated at the same temperature in both the prostate cell lines, showing a decrease in expression relative to the non-heated cells (FIGS. 2C, 2D, 2G and 2H). FIGS. 3A-3B show a Western blot demonstrating the expression of heat shock protein HSPA1A, as well as the cold shock proteins RBM3 and CIRBP, in PC-3 and LNCaP prostate cancer cells. While the amount of protein expression for the heat shock protein increased at 41° C. relative to 37° C., the amount of expression of both of the cold shock proteins decreased with heating. The level of actin expression is shown as a control. Similar experiments are performed to access the response of the tissues/cells to other environmental stresses.

Utilizing the methods described herein, tissues in which cold shock proteins are down-regulated in response to an environmental stress, such as heating of the tissue, including the application of mild hyperthermia, can be easily determined. The tissues can be removed from a patient, e.g., via a biopsy, and then stresses, e.g., heated to an appropriate temperature. Alternatively, the tissue can be stressed without removing it from the patent. Following the stress, such as heating (e.g., for about 4-12 hours at 39° C. to 41° C.), the expression of cold shock proteins can be assayed, for example, by either assaying for the level of expression of mRNA and/or of the proteins directly. The level of expression is then compared to the level of expression in tissue (e.g., another portion of the biopsied tissue), that has not been stressed, for example has not been heated (i.e., that has been maintained at about 37° C.). As described herein, a decrease in the level of expression of one or more cold shock proteins in a stressed (e.g., heated) tissue relative to an unstressed (e.g., unheated) tissue sample can be readily observed, for example, qualitatively by using a Western blot as shown in FIGS. 3A-3B, as well as other methods described herein and known in the art. This allows then for the identification of a tissue as one in which cold shock proteins are down-regulated in response to the stress (e.g., heat), and hence, as a tissue that is more susceptible to additional therapies, such as radiation, chemotherapy, etc., when combined with the environmental stress hyperthermia).

Various methods for heating tissues can be utilized in the practice of the present invention, and include the use of non-ionizing electromagnetic radiation, ionizing radiation or sound energy. Exemplary heating systems employ radio-frequency (RF) hyperthermia, such as annular Phased array systems (APAS), to tune E-field energy for regional heating of deep-seated tumors. Another strategy that utilizes RF hyperthermia requires surgical implantation of microwave- or RF-antennae or self-regulating thermal seeds. Additional methods for heating the tissues in the practice of the present invention include the use of microwave radiation, ultrasound, as well as implantable heating elements, such as heating catheters and the like.

In further embodiments, the tissues can be heated by administering one or more magnetic materials, suitably magnetic nanoparticles, to the patient, and then applying an alternating magnetic field to the materials (nanoparticles), so as to inductively heat the materials. Exemplary magnetic nanoparticles and methods of heating using the nanoparticles are disclosed in U.S. Pat. No. 7,074,175, the disclosure of which is incorporated by reference herein in its entirety for all purposes. Suitably, the magnetic nanoparticles comprise iron-containing materials, such as iron oxide, including superparamagnetic iron oxide, or maganese alloys, including alloys of the formula RMn₂X, where R is a rare earth metal, such as La, Ce, Pr or Nb, and X is either Ge or Si. The magnetic nanoparticles can be coated, for example with a synthetic or biological polymer, copolymer or polymer blend, or inorganic material, such as disclosed in U.S. Pat. No. 7,074,175. The nanoparticles can also comprise a targeting ligand, including ligands suitable for targeting cancer markers on cells. Suitable ligands, include, for example, proteins, peptides, antibodies, antibody fragments, saccharides, carbohydrates, glycans, proteoglycans, cytokines, chemokines, nucleotides, lectins, lipids, receptors, steroids, neurotransmitters, Cluster Designation/Differentiation (CD) markers, and imprinted polymers and the like. The ligands can be bound covalently or by physical interaction directly to an uncoated portion of the magnetic nanoparticle or to a coated portion of the nanoparticle.

Examples of ligands for attachment to the magnetic nanoparticles include Prostate-Specific Membrane Antigen (PSMA) and the EPCa-2 and EPCa-4 antigens, PSMA is an attractive target for targeting the nanoparticles to prostate cancer cells. Prostate-specific membrane antigen levels are markedly enhanced on the surface of advanced human prostate cancer cells that have failed androgen deprivation therapy. Similarly, the expression of the surface markers CD44, integrin α2β1 and CD133 characterize tumorigenic prostate cancer stem cells. Tumor-specific antibodies and aptamers have been developed to bind specifically to prostate-specific membrane antigen and stem cell antigens. Adding these binding agents to nanoparticles allow for specific heating of cancer cells.

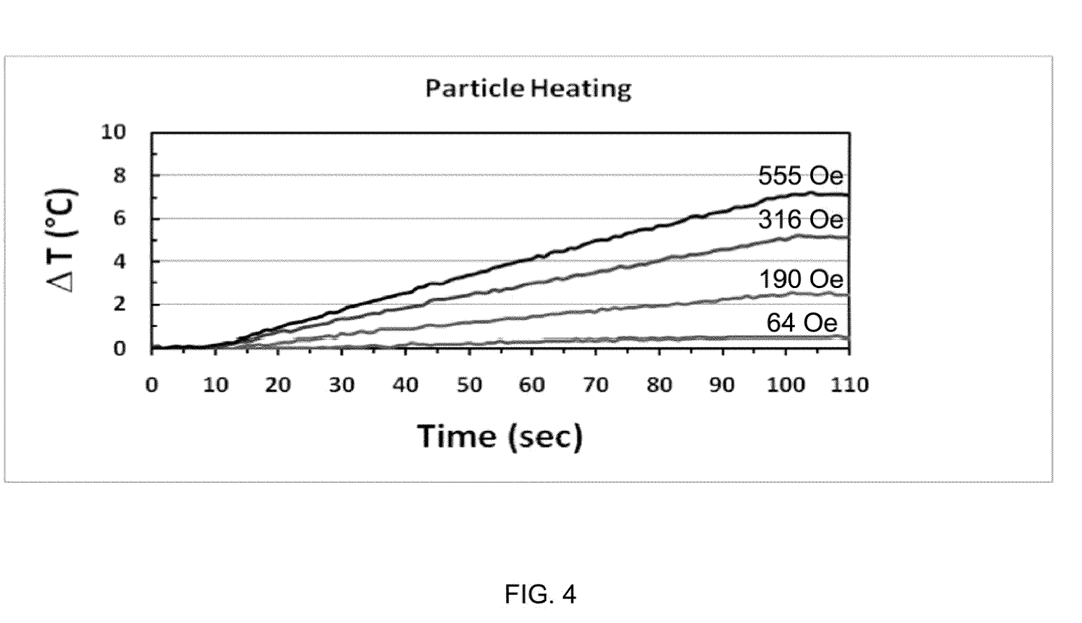

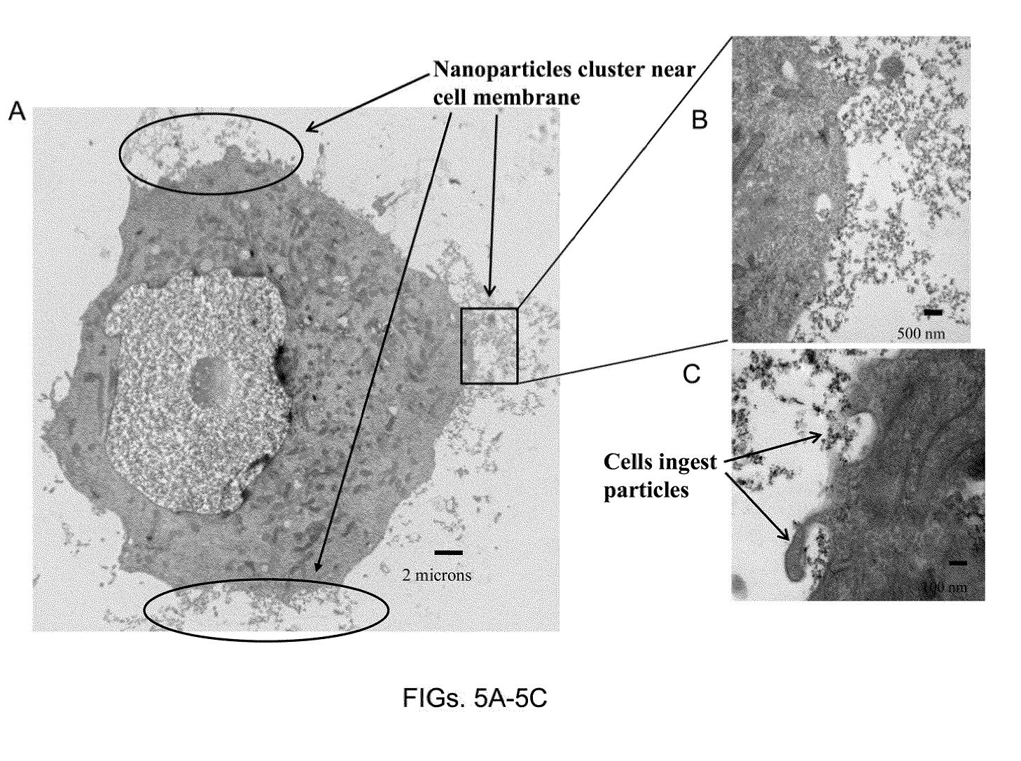

The temperature attained by heating utilizing magnetic nanoparticles can be tailored by selecting the appropriate nanoparticle size, as well as magnetic field strength. FIG. 4 shows the temperature increase obtained when exposing 10 mg/ml solutions of 100 nm magnetic nanoparticles to varying magnetic field strengths. An increase on the order of a few tenths of a degree Celsius can be obtained, up to about 7° C. (or more), thus clearly covering the range of about 39° C. to about 41° C., which is about 2-4° C. above 37° C. body temperature. As shown in FIGS. 5A-5C, nanoparticles targeted to prostate cancer cell using the Prostate-Specific Membrane Antigen (PSMA) ligand cluster near the cell membrane and are taken up by the cells (FIG. 5C). Thus, application of a magnetic field to the patient would cause an increase in temperature only at the local site of the tumor.

In further embodiments, the present invention provides methods of treating a patient suffering from a diseased tissue. The methods suitably comprise administering one or more nucleic acid molecules to the diseased tissue of the patient that down-regulate one or more cold shock proteins in the diseased tissue, wherein the susceptibility of the diseased tissue to an additional therapy is enhanced relative to diseased tissue which has not been administered the one or more nucleic acids.

As used herein, “diseased tissue” means a tissue that has been changed, damaged, infected or otherwise modified so as to he different than tissue of the same origin that has not been so modified. Examples of diseased tissues include cancerous tissues, virally or bacterially infected tissues, genetically mutated tissues, etc. Exemplary cancerous tissues that can he treated using the methods of the present invention include cancerous tissue of the heart, lung, breast, prostate, bladder, pancreas, brain, stomach, kidney, testes, lymph node, skin, bone, bone marrow, etc.

Methods for administering nucleic acid molecules that down-regulate one or more cold shock proteins in the diseased tissue are well known in the art. For example, the nucleic, acid molecules can be administered utilizing various vectors and/or carriers, including viral and non-viral vectors, plasmids, liposomes, polymers, etc. The nucleic acid molecules can be administered intravenously, systemically, orally, topically, subdermally, subcutaneously, intramuscularly, intratumoraly, etc.

In exemplary embodiments, the nucleic acid molecules which are administered are short interfering RNA (siRNA) molecules, though in additional embodiments, the nucleic acid molecules can be antisense nucleic acids. siRNA are 21-25 nucleotide RNA duplexes with a characteristic structure of a symmetric 2 nucleotide 3′-end overhang and a 5′ phosphate and 3′ hydroxy group. They were first identified in plants and Drosophila where they were associated with sequence specific inhibition of gene expression (Reviewed in Schutze, N. “siRNA Technology,” Molecular & Cellular Endocrinology 213: 115-119 (2004); and Scherr, M., Morgan, M. A., and Eder, M. “Gene silencing mediated by small interfering RNAs in mammalian cells.” Current Medicinal Chemistry 10: 245-256 (2003) the disclosures of which are incorporated herein by reference). Suitably, the nucleic acid molecules are siRNA that down-regulate cold shock proteins RBM3 and/or CIRBP. Sequences that can be used to down-regulate the cold shock proteins are described herein. Additional nucleic acid sequences, including mutations and variants that do not negatively effect the down-regulation of the target cold shock proteins, can also be utilized.

As described herein, down-regulation of one or more cold shock proteins in a diseased tissue increases the susceptibility of the diseased tissue to an additional therapy, relative to diseased tissue which has not been administered one or more nucleic acids that down-regulate one or more cold shock proteins. “Susceptibility” as used herein, refers to the response of a diseased tissue to a therapy, or the effect of a therapy on the tissue (i.e., the ability of a therapy to kill or slow the growth of cancer cells, or otherwise treat the diseased tissue). As used herein, “increased susceptibility” is used to indicate that the response of a diseased tissue to an additional therapy is enhanced (i.e., the additional therapy is more effective at killing or slowing the growth of the cancer cells, or otherwise treating the diseased tissue) relative to a tissue that has not been heated. It has been determined that down-regulation of cold shock proteins results in a response in the tissue that is similar to that when the tissue is environmentally stressed (e.g., heated). Thus, as the susceptibility of the diseased tissue to additional therapies is increased following an environmental stress (e.g., heating at mild-hyperthermic temperatures), the down-regulation of cold shock proteins also causes an increase in the susceptibility of the tissues, seeming to mimic the effect of the stress (e.g., heating).

Thus, in further embodiments, the methods suitably further comprise administering an additional therapy to the diseased tissue, in addition to the down-regulation of the cold shock proteins. Suitable additional therapies include, but are not limited to, administering a chemotherapeutic agent, administering radiation therapy, administering immunotherapy, administering radio-immunotherapy, administering gene therapy and/or administering gene silencing therapy, to the patient. Exemplary chemotherapeutic agents that can be administered include, but are not limited to, methotrexate, adriamycin, epirubicin, daunorubicin, doxorubicin, amphotericin B, vincristine, vinblastine, etoposide, ellipticine, camptothecin, paclitaxel, docetaxol, cisplatin, prednisone, methyl-prednisone, and navalbene.

Methods for administering the additional therapies are well known in the art, as are protocols for the administrations. In exemplary embodiments, the additional therapy is administered after the administration of the nucleic acid molecules to down-regulate the cold shock proteins, for example, the additional therapy can be administered hours, days or weeks after the administration of the nucleic acid molecules. In other embodiments, the nucleic acid molecules are administered after the administration of the additional therapy has started, and the additional therapy is then continued following the down-regulation of the cold shock proteins. In further embodiments, both the down-regulation of the cold shock proteins and the additional therapies can be started at the same time. The preparation of appropriate protocols for patient dosing and timing can be easily determined by those of ordinary skill in the art. In certain embodiments, more than one additional therapies can be administered to the subject (e.g., administration of a chemotherapeutic agent and radiation therapy, etc). The additional therapies can be administered at the same time, or can be administered at different times, according to well known clinical protocols.

For example, in embodiments where the additional therapy comprises the administration of one or more chemotherapeutic agents, administration of the nucleic acid molecules to down-regulate the cold shock proteins suitably occurs prior to administration of the chemotherapeutic agent (e.g., minutes, hours, days, weeks, etc., before the chemotherapeutic agent). In additional embodiments, the down-regulation can occur just prior to administration of the chemotherapeutic agent, or it can occur after the chemotherapeutic agent has been administered, or during the administration of the chemotherapeutic agent (for example, during an intravenous drip or injection). In embodiments where the additional therapy comprises administration of radiation therapy, suitably the radiation is administered alter the down-regulation of the cold shock proteins.

In additional embodiments, the methods further comprise administering one or more nucleic acid molecules to the patient's tissue that down-regulate one or more heat shock proteins in the tissue. The nucleic acid molecules can he antisense molecules, or suitably siRNA. Various heat shock proteins which can be down-regulated are known in the art, including heat shock 70 kDa protein IA (HSPA1A), heat shock 70 kDa protein 1-like (HSPA1L), heat shock 105 kDa/110 kDa protein 1 (HSPA1), heat shock 70 kDa protein 4-like (HSPA4L), heat shock 70 kDa protein 5 (HSPA5), heat shock 27 kDa protein 1 (HSPB1), heat shock protein 90 kDa alpha, class A member 2 (HSP90AA2), AHA 1, activator of heat shock 90 kDA protein ATPase homolog 1 (AHSA1). Sequences suitable for down-regulation of heat shock proteins are well known or can be designed, and are readily developed and prepared by those of ordinary skill in the art (see e.g., Frese et al., Journal of Thoracic and Cardiovascular Surgery 126: 748-754 (2003); Hosaka et al., Cancer Science 97:623-632 (2006); Friedman, Nature Biotechnology 26: 399-400 (2008); Hagiwara et al, Respiratory Research 8:37 (2007); Hadaschik et al., British Journal of Urology 102:610-616 (2008); and McGarry and Lindquist, Proceedings of the National Academy of Sciences 83:399-103 (1986), the disclosures of each of which are incorporated by reference herein in their entireties.

In additional embodiments, small molecule therapeutics that act to reduce the heat shock response can also be administered in the various methods described herein. Examples of small molecules that can be administered in the various methods include 5-aminoimidazole-4-carboxamide-1-β-D-ribofuranoside (AICAR), shepherdin, etc.

The methods of the present invention are suitably used to treat mammalian patients, such as a dog, cat, horse, pig, mouse, rat, goat, or primate. Suitably the methods are used to treat human patients, including human cancer patients.

In further embodiments, the present invention provides additional methods of treating a patient (e.g., a mammal, such as a human) suffering from a diseased tissue. The methods suitably comprise environmentally stressing the diseased tissue of the patient for a period of greater than about 10 minutes. Suitably, the tissue is locally heated, so as to raise the temperature of the diseased tissue to about 39° C. to about 41° C. for a period of greater than about 10 minutes. One or more nucleic acid molecules are administered that down-regulate one or more cold shock proteins in the diseased tissue, to the patient. An additional therapy is administered to the patient, wherein the susceptibility of the diseased tissue to the additional therapy is enhanced relative to diseased tissue which has not been heated and administered one or more nucleic acids that down-regulate one or more cold shock proteins.

As used herein, the term “locally heating” means that the temperature of the diseased tissue, for example tumor tissue, is raised to a mild hyperthermic level (e.g., about 39° C. to about 41° C.), but that surrounding tissue (including normal, non-diseased tissue) is not substantially raised above normal temperature. Suitably, for a human, the temperature of the surrounding tissue is not raised above about 37-38° C. As used herein “surrounding tissue” means tissue outside of a distance of about 5-10 mm from the diseased tissue. As described herein, suitably the temperature of the diseased tissue is raised for a period of about 30 minutes to about 24 hours, about 1 hour to about 12 hours, about 4 hours to about 8 hours, or about, 1 hour, about 2 hours, about 3 hours, about 4 hours, about 5 hours, about 6 hours, about 7 hours, about 8 hours, about 9 hours, about 10 hours, about 11 hours or about 12 hours. In exemplary embodiments, the temperature of the diseased tissue is raised to a temperature of about 39° C. to about 41° C., and any temperature or range of temperatures between 39° C. to about 41° C. Suitably, the heating is to a temperature of about 39° C. to about 40° C., about 40° C. to about 41° C., or about 39° C., about 39.5° C., about 40° C., about 40.5° C., or about 41° C.

Exemplary methods of locally heating the diseased tissue are described herein and known in the art. Suitably, the local heating comprises application of non-ionizing electromagnetic radiation, ionizing radiation or sound energy. In further embodiments, as described herein, the local heating comprises administering a magnetic material (e.g., nanoparticles) to the patient and applying an alternating magnetic field so as to inductively heat the magnetic material.

The environmental stressing (e.g., local heating) of the diseased tissue and the administration of the nucleic acid molecules can occur in any order, for example, the tissue can be stressed (e.g., heated), and then the nucleic acid molecules can be administered, alternatively the nucleic acid molecules can be administered followed by stressing (e.g., heating). In embodiments where the stressing is started before the administration of the nucleic acid molecules, suitably the stressing is maintained at least until the administration begins, and suitably, throughout the administration of the nucleic acid molecules. For example, the diseased tissue can be stressed, and then the administration started (e.g., an intravenous injection or slow drip), followed by further stressing. In still further embodiments, the stressing and the administration of the nucleic acid molecules can occur at the same, or substantially the same time.

Exemplary therapies that can be administered in addition to the stressing (e.g., heating) and down-regulation of the cold shock proteins are described herein, and include, but are not limited to, administering a chemotherapeutic agent, administering radiation therapy, administering immunotherapy, administering radio-immunotherapy, administering gene therapy and/or administering gene silencing therapy, to the patient. Suitably, the chemotherapeutic agent comprises administering an agent selected from the group consisting of methotrexate, adriamycin, epirubicin, daunorubicin, doxorubicin, amphotericin B, vincristine, vinblastine, etoposide, ellipticine, camptothecin, paclitaxel, docetaxol, cisplatin, prednisone, methyl-prednisone, and navalbene. As described herein, more than one additional therapy cats be administered using well known clinical protocols.

The administration of the additional therapy, the environmental stressing (e.g., local heating) of the diseased tissue, and the administration of the nucleic acid molecules, can occur in any order. For example, the tissue can be stressed (e.g., heated), and then the nucleic acid molecules can be administered, followed by the administration of the additional therapy. Alternatively, the nucleic acid molecules can be administered followed by stressing (e.g., heating), and subsequently the additional therapy can be administered. Suitably, the stressing is maintained during the administration of the additional therapy. For example, the diseased tissue can be stressed, and then the administration of the additional therapy started, followed by further stressing. In still further embodiments, the stressing (e.g., heating) and the administration of the additional therapy can occur at the same, or substantially the same time. Suitably, the administration of the nucleic acid molecules will occur prior to any stressing and/or administration of an additional therapy so that the cold shock proteins can be sufficiently down-regulated, thereby allowing for the enhanced susceptibility.

In additional embodiments, as described herein, the methods further comprise administering one or more nucleic acid molecules (including siRNA and antisense molecules) to the tissue that down-regulate one or more heat shock proteins in the tissue, as well as small molecules that interfere with the heat shock response. Exemplary heat shock proteins that can be down-regulated are described herein and well known in the art. In addition, nucleic acid sequences that can be used to down-regulate the heat shock proteins are readily determined by those of ordinary skill in the art.

In still further embodiments, the present invention provides additional methods of treating a patient suffering from a diseased tissue. The methods suitably comprise identifying a diseased tissue of the patient in which cold shock proteins are down-regulated in response to stressing (e.g., heating) of the diseased tissue. The diseased tissue is then stressed. For example, the tissue is locally heated, so as to raise the temperature of the tissue to about 39° C. to about 41° C., suitably for a period of greater than about 10 minutes. As described herein the susceptibility of the diseased tissue to an additional therapy is enhanced relative to unheated diseased tissue.

Exemplary methods for identifying a diseased tissue of the patient in which cold shock proteins are down-regulated in response to stressing (e.g., heating) are described herein, and include analysis of protein and/or RNA expression levels using methods well known in the art. As described herein, such methods can be quantitative and/or qualitative.

As described throughout, suitably the diseased tissue is a cancerous tissue, such as a cancerous tissue of the heart, lung, breast, prostate, bladder, pancreas, brain, stomach, kidney, testes, lymph node, skin, bone, bone marrow, etc. Methods for locally heating the diseased tissue, as well as temperatures and times for the heating are described throughout.

In suitable embodiments, the methods further comprise administering to the patient one or more additional therapies selected from the group consisting of administering a chemotherapeutic agent, administering radiation therapy, administering immunotherapy, administering radio-immunotherapy, administering gene therapy and administering gene silencing therapy. The order of administration of the additional therapy relative to the down-regulation of cold shock proteins and stressing (e.g., heating) of the tissue are described herein. As described herein, more than one additional therapy can be administered using well known clinical protocols. In additional embodiments, one or more nucleic acid molecules can be administered to the tissue that down-regulate one or more heat shock proteins in the tissue.

As described throughout, suitably the nucleic acid molecules for down-regulation of the cold shock and heat shock proteins are antisense or siRNA. Exemplary cold shock and heat shock proteins that can be down regulated are described throughout.

It will be readily apparent to one of ordinary skill in the relevant arts that other suitable modifications and adaptations to the methods and applications described herein may he made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLE 1

### Down-Regulation of Cold Shock Proteins for Cancer Therapy

### Materials & Methods

Cell culture and treatment: Prostate cancer cell lines PC-3 and LNCaP were obtained from the American Type Culture Collection (Rockville, Md.). The cells were cultured at 37° C. in routine RPMI (Invitrogen, Carlsbad. Calif.) media supplemented with 10% fetal bovine serum (FBS) and treated by mild heat for an indicated duration in an incubator at 41° C., or simultaneously heated to 41° C. and treated with chemical drug for 4 h. Controls, including chemically treated and untreated samples, were maintained at 37° C.

RNA isolation, microarray experiments and data analysis: Total RNA isolation, fragmentation and microarray hybridization and scanning procedures are carried out following the suppliers protocol (Agilent Technologies). Lowess normalization was used to normalize the intensity log ratio M of the non-control probes. All computations were performed under R environment (The R Foundation for Statistical Computing).

Reverse transcription and real-time PCR: RNA samples were treated with DNase I (Invitrogen, Carlsbad, Calif.), and cDNA was synthesized using the iScript™ cDNA synthesis kit (BioRad, Hercules, Calif.). Real-time PCR was done in triplicate on an iCycler iQ™ Multicolor Real-time PCR Detection system (BioRad). Target gene expression was related to TATA box binding protein (TBP) for normalization. PCR sequences used were shown in Table 1 below.

TABLE 1
Primer sequences used in real-time PCR

| Gene name | Primer sequences |
|---|---|
| HSPA1A | Forward: 5′-CAGGTGATCAACGACGGAGACA-3′ (SEQ ID NO: 1) |
| | Reverse: 5′-GTCGATCGTCAGGATGGACACG-3′ (SEQ ID NO: 2) |
| HSPA6 | Forward: 5′-CCGTGAAGCACGCAGTGAT-3′ (SEQ ID NO: 3) |
| | Reverse: 5′-ACGAGCCGGTTGTCGAAGT-3′ (SEQ ID NO: 4) |
| RBM3 | Forward: 5′-CTTCAGCAGTTTCGGACCTA-3′ (SEQ ID NO: 5) |
| | Reverse: 5′-ACCATCCAGAGACTCTCCGT-3′ (SEQ ID NO: 6) |
| CIRBP | Forward: 5′-CAAAGTACGGACAGATCTCTGA-3′ (SEQ ID NO: 7) |
| | Reverse: 5′-CGGATCTGCCGTCCATCTA-3′ (SEQ ID NO: 8) |
| TATABP | Forward: 5′-GAATATAATCCCAAGCGGTTTG-3′ (SEQ ID NO: 9) |
| | Reverse: 5′-ACTTCACATCACAGCTCCCC-3′ (SEQ ID NO: 10) |

Western blotting: Twenty-five micrograms of protein were separated on 10% to 20% SDS-PAGE and transferred onto PVD filters (Millipore, Bedford, Mass.). Membranes were incubated with primary antibodies overnight at 4° C. followed by horseradish peroxidase-conjugated secondary antibodies, and developed with the Super Signal West Dura Extended Duration Substrate kit (Pierce). RBM3 antibody was generated in a rabbit against as peptide (Sigma-Gneosys, The Woodlands, Tex.). HSPA1A and CIRBP antibodies were obtained from Lifespan Biosciences (ProteinTech Group, Chicago, Ill.), phospho-Histone H2A.X antibody was obtained from Millipore. Other antibodies were purchased from Cell Signaling (Danvers, Mass.) and Santa Cruz (Santa Cruz, Calif.)

Nuclear matrix protein isolation, two-dimensional gel electrophoresis and protein identification: Nuclear matrix protein extraction and high resolution, two-dimensional electrophoresis was performed as previously described (Inoue et al. 2008). Protein identification was done by LC matrix-assisted laser desorption/ionization mass spectrometry (LC/MALDI MS) using an ABI Tempo LC MALDI mass spectrometer in the reflector mode using delayed extraction. Peptides were analyzed by collision-induced dissociation (CID) using nano LC tandem mass spectrometry analysis on a LTQ (Thermo Fisher Scientific, Waltham, Mass.) (Shevchenko et al., 1996). Peptide sequences were identified by screening the fragmentation data against the NCBI non-redundant database (uniprot_sprot_{—}20070123) using the Mascot sequence query search engine (Matrix Science, Boston, Mass.). Identified sequences were confirmed by manually inspecting CID spectra.

Cell viability inhibition assay: Five thousand cells per well were seeded in 96-well plates. Seventy-two hours after drug treatment, cell proliferation reagent WST-1 (4-[3-(4-iodophenyl)-2-(4-nitrophenyl)-2H-5-tetrazolio]-1,3-benzene disulfonate) was added to each well, as specified by the supplier (Roche, Nutley, N.J.). After a 4-h incubation, WST-1 absorbance at 450 nm was measured.

siRNA transfections: RBM3 and CIRBP ON-TARGETplus™ ” SMARTpool™ siRNA were obtained from Dharmacon (Lafayette, Colo.), the sequences of which are set forth below, 2.5×10³ cells or 5×10³ LNCaP cells were transfected with siRNA at 100 nM total oligo concentration, using 0.2 μl DharmaFECT-3 transfection reagent, and plated simultaneously in a 96-well plate. Chemical treatment was conducted 24-48 h after transfection.

| siRNA for CIRBP | |
|---|---|
| GGUCCUACAGAGACAGUUA | (SEQ ID NO: 11) |
| GAACAUUGACGACGCUAAG | (SEQ ID NO: 12) |
| GAGCAGGUCUUCUCAAAGU | (SEQ ID NO: 13) |
| CCAGAGAUCUCGGGGAUUU | (SEQ ID NO: 14) |
| siRNA for RBM3 | |
| ACAUGAUUAUCCAGCGGUA | (SEQ ID NO: 15) |
| AGACAAAUUAUGGGACGUU | (SEQ ID NO: 16) |
| GUGGGAGGGCUCAACUUUA | (SEQ ID NO: 17) |
| GGAUCAUGCAGGCAAGUCU | (SEQ ID NO: 18) |

FACS analysis of apoptosis and cell cycle: Apoptosis and cell cycle were detected on a GUAVA® system (Guava Technologies, Hayward, Calif.) using TUNEL kit (Guava) and GUAVA® Cell Cycle Reagent (Guava), respectively.

Clonogenic assays: PC-3 and LNCaP cells were transfected with 100 nM RBM3 or CIRBP siRNA. Forty-eight hours after transfection, cells were plated at 500 cells in 10-cm dish and grown without being disturbed. Colonies were counted after 2 weeks. Only colonies containing more than 50 cells were included.

Statistical analysis: Comparisons were made using the Student's t test. Two-sided P of less than 0.05 was considered significant.

### Results

In order to elucidate the changes in cellular pathways in response to mild heat, an experimental design which focuses on a single mild temperature increase was implemented (FIG. 1). Other in vitro studies have demonstrated that 1-4 h of heating (depending on the sequence of modalities) at 41° C. produces substantial heat-induced radiosensitization with little or no cell killing by heat alone (10). This temperature is also one which is clinically feasible. The androgen-responsive LNCaP prostate cancer cells and the aggressive androgen-insensitive PC-3 cancer cells representing relatively diverse forms of the disease were utilized. Cells were grown at the normal temperature of 37° C. or at 41° C. to discern their response to heat-shock for the designated time periods (FIG. 1).

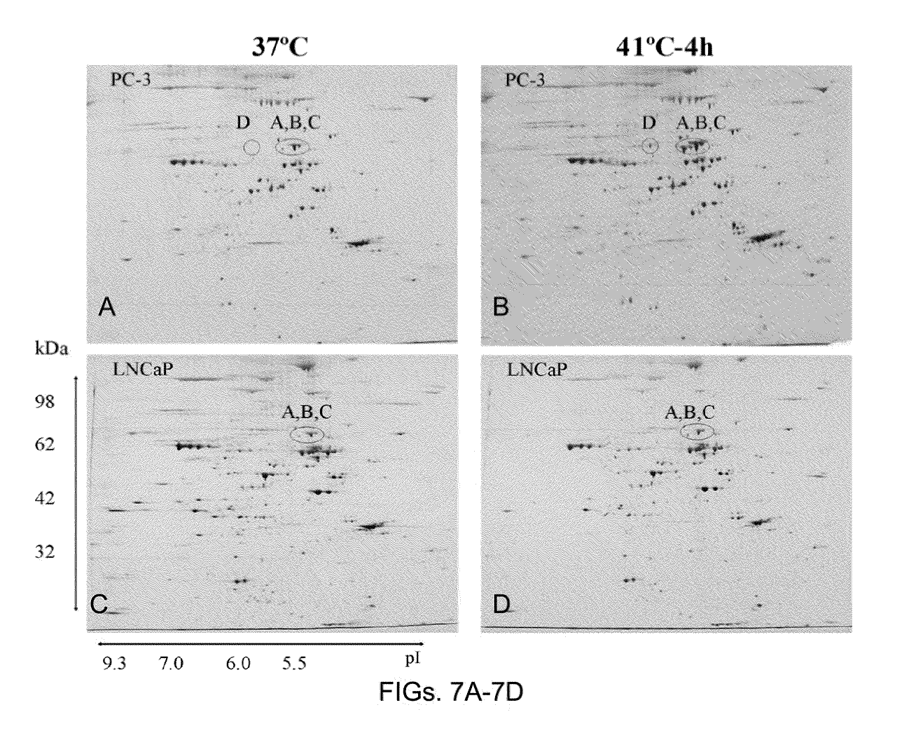

Changes in Nuclear Matrix Protein composition in response to heat: As changes in nuclear matrix protein (NMP) composition have often been accompanied by changes in cellular pathology, proteomic analysis of the nuclear matrix preparations from both cell lines was performed. Several spots appeared to be distinctly over-expressed upon heat shock that were identified by mass-spectrometric peptide sequencing as heat shock 70 kDa protein 1A (Hspa1a), heat shock 70 kDa protein 8 (Hspa8) and heat shock 70 kDa protein 6 (Hspa6), as shown in FIG, 6. However, no protein spots were observed that appeared to be significantly down-regulated upon heat treatment at 41° C., as shown in FIGS. 7A-7D.

FIGS. 7A-7D show the mapping of nuclear matrix proteins (NMPs) changes in response to heat. Prostate cancer cells PC-3 and LNCaP were cultured at 41° C. for 4 h or maintained at 37° C. as a control. NMPs were isolated and then subjected to high-resolution, two-dimensional gel electrophoresis followed by a silver staining. The differential expression of the NMPs upon heat treatment was cut out and subjected to mass spectrometry (LC/MALDI MS) analysis. Differential proteins are labeled and marked by circles. All experiments were repeated a minimum of two times.

Mild heat treatment up-regulates heat shock proteins but down-regulates cold shock proteins: In order to correlate the changes in protein expression with concomitant changes in gene expression, global gene expression profiles were interrogated using DNA microarrays. Only those genes that had odds ratios of ≧10 and a fold change of ≧1.5 were selected as being significantly different. In LNCaP and PC-3 cells, 216 and 158 genes are dysregulated, respectively (Tables 2 and 3 below). Of the dysregulated genes, 54 genes are shared by both cell lines (Table 4) suggesting that there may be common pathways that cancer cells utilize to counter the stress response. The majority of the differentially regulated genes are up-regulated genes in both cell lines. Of these, as one would expect, the most abundant genes are those encoding the heat shock proteins (HSPs) and proteins related to modulating HSPs such as AHSA1, corroborating the results from the proteomic analysis. Among other classes of genes are those that encode transcription factors such as CAMTA2 and WBP5, transporters such as SLC1A3, and those related to apoptosis such as BAG3 and THAP2. Interestingly, unlike the proteomic analysis, gene expression profiling also revealed genes that are significantly down-regulated including those encoding the RNA-binding proteins, many of which belong to the ‘cold shock protein’ family such as cold inducible RNA binding protein (CIRBP) and RNA binding motif protein 3 (RBM3) (Table 4).

TABLE 2
Dysregulated gene list in PC-3 cells upon heat treatment*

| Systematic name | Gene name | Fold change to 37° C. |
|---|---|---|
| NM_005345 | HSPA1A | 7.7 |
| NM_005527 | HSPA1L | 6.0 |
| NM_017510 | TMED9 | 4.1 |
| XM_001133211 | LOC729003 | 3.0 |
| NM_020435 | GJA12 | 2.9 |
| NM_006644 | HSPH1 | 2.8 |
| NM_000916 | OXTR | 2.6 |
| NM_004281 | BAG3 | 2.5 |
| NM_017742 | ZCCHC2 | 2.5 |
| BC006998 | BC006998 | 2.5 |
| NM_014278 | HSPA4L | 2.4 |
| NM_003325 | HIRA | 2.3 |
| NM_020317 | C1orf63 | 2.3 |
| BC010426 | BC010426 | 2.3 |
| AB028966 | TTC28 | 2.3 |
| NM_012124 | CHORDC1 | 2.2 |
| NR_002802 | TncRNA | 2.2 |
| NM_004199 | P4HA2 | 2.2 |
| AK022110 | AK022110 | 2.2 |
| AJ001827 | AJ001827 | 2.1 |
| NM_130469 | JDP2 | 2.1 |
| NM_007034 | DNAJB4 | 2.1 |
| AK021552 | LOC399959 | 2.1 |
| NM_001114 | ADCY7 | 2.1 |
| CR616003 | CR616003 | 2.1 |
| NM_206956 | PRAME | 2.1 |
| NM_001539 | DNAJA1 | 2.1 |
| AK094167 | FLJ36848 | 2.0 |
| XR_018209 | LOC647907 | 2.0 |
| A_24_P401090 | A_24_P401090 | 2.0 |
| NM_001321 | CSRP2 | 2.0 |
| NM_199133 | LOC134145 | 2.0 |
| XR_017030 | LOC653364 | 2.0 |
| A_32_P85880 | A_32_P85880 | 2.0 |
| NM_005347 | HSPA5 | 2.0 |
| BC065040 | C1orf63 | 2.0 |
| NM_001540 | HSPB1 | 1.9 |
| ENST00000378770 | ENST00000378770 | 1.9 |
| NM_015107 | PHF8 | 1.9 |
| NM_030641 | APOL6 | 1.9 |
| THC2705456 | THC2705456 | 1.9 |
| NM_022802 | CTBP2 | 1.9 |
| NM_003451 | ZNF177 | 1.9 |
| THC2650074 | THC2650074 | 1.9 |
| NM_054021 | GPR101 | 1.9 |
| NM_032160 | C18orf4 | 1.9 |
| NM_018357 | LARP6 | 1.9 |
| NM_001040141 | HSP90AA2 | 1.9 |
| NM_004172 | SLCLA3 | 1.8 |
| A_32_P171043 | A_32_P171043 | 1.8 |
| NM_006145 | DNAJB1 | 1.8 |
| NM_006010 | ARMET | 1.8 |
| NM_018602 | DNAJA4 | 1.8 |
| BC033366 | KRT77 | 1.8 |
| NM_005931 | MICB | 1.8 |
| NM_000481 | AMT | 1.8 |
| NM_012111 | AHSA1 | 1.8 |
| NM_015099 | CAMTA2 | 1.8 |
| NM_172037 | RDH10 | 1.8 |
| AK096054 | SLC6A19 | 1.8 |
| NM_005348 | HSP90AA1 | 1.8 |
| THC2521437 | THC2521437 | 1.7 |
| NM_207435 | FLJ40142 | 1.7 |
| U60873 | TncRNA | 1.7 |
| NM_001005199 | OR8H1 | 1.7 |
| NM_144778 | MBNL2 | 1.7 |
| NM_198951 | TGM2 | 1.7 |
| A_24_P914208 | A_24_P914208 | 1.7 |
| NM_001868 | CPA1 | 1.7 |
| NM_000539 | RHO | 1.7 |
| NM_005573 | LMNB1 | 1.7 |
| NM_052957 | ACRC | 1.7 |
| NM_006736 | DNAJB2 | 1.7 |
| NM_007184 | NISCH | 1.7 |
| NM_001008397 | LOC493869 | 1.7 |
| NM_002154 | HSPA4 | 1.7 |
| NM_017633 | FAM46A | 1.7 |
| NM_182972 | IRF2BP2 | 1.7 |
| A_32_P220161 | A_32_P220161 | 1.7 |
| AI732190 | AI732190 | 1.7 |
| AK123861 | AK123861 | 1.7 |
| NM_002133 | HMOX1 | 1.7 |
| NM_001017973 | P4HA2 | 1.7 |
| NM_138931 | BCL6 | 1.7 |
| AK095791 | LOC399959 | 1.7 |
| BC041467 | C17orf67 | 1.7 |
| NM_031435 | THAP2 | 1.7 |
| AF095737 | SARDH | 1.7 |
| NM_003315 | DNAJC7 | 1.7 |
| NM_199040 | NUDT4 | 1.7 |
| NM_004609 | TCF15 | 1.7 |
| NM_016303 | WBP5 | 1.6 |
| NM_003124 | SPR | 1.6 |
| NM_014384 | ACAD8 | 1.6 |
| AL050061 | LOC157562 | 1.6 |
| NM_015513 | CRELD1 | 1.6 |
| THC2739159 | THC2739159 | 1.6 |
| THC2654987 | THC2654987 | 1.6 |
| AK024095 | HIF3A | 1.6 |
| NM_006361 | HOXB13 | 1.6 |
| NM_001172 | ARG2 | 1.6 |
| THC2669063 | THC2669063 | 1.6 |
| XR_015848 | LOC730211 | 1.6 |
| ENST00000310492 | MGA | 1.6 |
| NM_000164 | GIPR | 1.6 |
| NM_014409 | TAF5L | 1.6 |
| NM_144608 | HEXIM2 | 1.6 |
| NM_019116 | UBPH | 1.6 |
| NM_024081 | PRRG4 | 1.6 |
| NM_032356 | LSMD1 | 1.6 |
| A_32_P222060 | A_32_P222060 | 1.6 |
| NM_020157 | OTOR | 1.6 |
| THC2611661 | THC2611661 | 1.6 |
| BC104151 | FLJ31306 | 1.6 |
| NM_002155 | HSPA6 | 1.6 |
| NM_001498 | GCLC | 1.6 |
| THC2653489 | THC2653489 | 1.6 |
| NM_001018062 | LOC51149 | 1.6 |
| AL359062 | AL359062 | 1.6 |
| NM_020338 | ZMIZ1 | 1.6 |
| AK002097 | FLJ11235 | 1.6 |
| NM_016626 | RKHD2 | 1.6 |
| BE168511 | BE168511 | 1.6 |
| NM_020704 | FAM40B | 1.6 |
| ENST00000371207 | ENST00000371207 | 1.6 |
| NM_006805 | HNRPA0 | 1.6 |
| NR_001543 | TTTY14 | 1.6 |
| A_32_P169243 | A_32_P169243 | 1.6 |
| AK023669 | CENPN | −1.6 |
| NM_014660 | PHF14 | −1.6 |
| NM_152904 | SPECC1 | −1.6 |
| NM_001280 | CIRBP | −1.6 |
| NM_001356 | DDX3X | −1.6 |
| ENST00000381577 | CD274 | −1.6 |
| NM_017858 | TIPIN | −1.6 |
| NM_005033 | EXOSC9 | −1.6 |
| NM_024854 | FLJ22028 | −1.7 |
| NM_032043 | BRIP1 | −1.7 |
| ENST00000222543 | TFP12 | −1.7 |
| NM_001017430 | RBM3 | −1.7 |
| NM_000576 | IL1B | −1.7 |
| NM_006979 | SLC39A7 | −1.7 |
| NM_205841 | SPINK6 | −1.7 |
| NM_194460 | RNF126 | −1.7 |
| NM_000584 | IL8 | −1.7 |
| NM_000600 | IL6 | −1.7 |
| BC063625 | KRTAP2-4 | −1.8 |
| NM_000358 | TGFB1 | −1.8 |
| NM_000227 | LAMA3 | −1.8 |
| NM_016584 | IL23A | −1.8 |
| NM_198129 | LAMA3 | −1.9 |
| NM_032292 | GON4L | −1.9 |
| NM_031372 | HNRPDL | −1.9 |
| NM_032865 | TNS4 | −2.0 |
| NM_153840 | GPRI10 | −2.1 |
| NM_002575 | SERPINB2 | −2.1 |
| NM_002658 | PLAU | −2.1 |
| NM_000963 | PTGS2 | −2.1 |

*All dysregulated genes were selected by Odd ratio ≧10, fold change ≧1.5.

TABLE 3
Dysregulated gene list in LNCaP cells upon heat treatment*

| Systematic name | Gene name | Fold change to 37° C. |
|---|---|---|
| NM_005345 | HSPA1A | 5.8 |
| NM_005527 | HSPA1L | 4.7 |
| NM_173561 | UNC5CL | 4.6 |
| NM_014685 | HERPUD1 | 4.6 |
| NM_207123 | GAB1 | 4.3 |
| NM_004281 | BAG5 | 3.8 |
| NM_007034 | DNAJB4 | 3.6 |
| NM_006644 | HSPH1 | 3.6 |
| NM_203497 | COMMD6 | 3.4 |
| NM_012328 | DNAJB9 | 2.9 |
| A_24_P401090 | A_24_P401090 | 2.8 |
| NM_004083 | DDIT3 | 2.7 |
| NM_005347 | HSPA5 | 2.7 |
| NM_006065 | SIRPB1 | 2.6 |
| NM_006145 | DNAJB1 | 2.6 |
| NM_014278 | HSPA4L | 2.4 |
| NM_012124 | CHORDC1 | 2.4 |
| NM_021806 | FAM3A | 2.4 |
| AK022110 | AK022110 | 2.3 |
| NM_198273 | LYSMD3 | 2.3 |
| NM_001001971 | FAM13C1 | 2.3 |
| NM_130469 | JDP2 | 2.2 |
| NM_006010 | ARMET | 2.2 |
| NM_004199 | P4HA2 | 2.2 |
| NM_004864 | GDF15 | 2.2 |
| NM_182491 | ZFAND2A | 2.2 |
| AK097398 | NUCB2 | 2.1 |
| NM_001539 | DNAJA1 | 2.1 |
| NM_016303 | WBP5 | 2.1 |
| NM_172230 | SYVN1 | 2.1 |
| NM_001956 | EDN2 | 2.1 |
| NM_001039492 | FHL2 | 2.1 |
| ENST00000378770 | ENST00000378770 | 2.1 |
| NM_000164 | GIPR | 2.0 |
| NM_004733 | SLC33A1 | 2.0 |
| NM_002155 | HSPA6 | 2.0 |
| NM_003447 | ZNF165 | 2.0 |
| NM_006948 | STCH | 2.0 |
| BC035106 | BC035106 | 1.9 |
| NM_003947 | KALRN | 1.9 |
| NM_000203 | IDUA | 1.9 |
| XR_017030 | LOC653364 | 1.9 |
| CR611122 | CR611122 | 1.9 |
| NM_001114 | ADCY7 | 1.9 |
| CP143262 | CF143262 | 1.9 |
| NM_005494 | DNAJB6 | 1.9 |
| THC2650074 | THC2650074 | 1.9 |
| NM_015513 | CRELD1 | 1.9 |
| NM_002167 | ID3 | 1.9 |
| XR_018209 | LOC647907 | 1.9 |
| NM_005194 | CEBPB | 1.9 |
| NM_021158 | TRIB3 | 1.8 |
| XR_015848 | LOC730211 | 1.8 |
| NM_024039 | MIS12 | 1.8 |
| NM_018372 | C1orf103 | 1.8 |
| AK022339 | AK022339 | 1.8 |
| NM_002165 | ID1 | 1.8 |
| NM_001040619 | ATF3 | 1.8 |
| NM_052957 | ACRC | 1.8 |
| NM_001017973 | P4HA2 | 1.8 |
| NM_005348 | HSP90AA1 | 1.8 |
| NM_032876 | JUB | 1.8 |
| XM_933296 | LOC645955 | 1.8 |
| NM_207435 | FLJ40142 | 1.8 |
| U50529 | RP11-298P3.3 | 1.7 |
| XM_001133211 | LOC729003 | 1.7 |
| NM_001040141 | HSP90AA2 | 1.7 |
| NM_014445 | SERP1 | 1.7 |
| NM_173516 | PNLDC1 | 1.7 |
| NM_007076 | HYPE | 1.7 |
| NM_005088 | CXYorf3 | 1.7 |
| NM_001008397 | LOC493869 | 1.7 |
| AF086329 | AF086329 | 1.7 |
| NM_033285 | TP53INP1 | 1.7 |
| NM_032229 | SLITRK6 | 1.7 |
| NM_182972 | IRF2BP2 | 1.7 |
| NM_006605 | RFPL2 | 1.7 |
| NM_018266 | TMEM39A | 1.7 |
| NM_199133 | LOC134145 | 1.7 |
| NM_032356 | LSMD1 | 1.7 |
| NM_005951 | MT1H | 1.7 |
| NM_005952 | MT1X | 1.7 |
| A_24_P16361 | A_24_P16361 | 1.7 |
| NM_004836 | EIF2AK3 | 1.7 |
| NM_005065 | SEL1L | 1.7 |
| NM_001540 | HSPB1 | 1.7 |
| NM_020317 | C1orf63 | 1.7 |
| NM_004172 | SLC1A3 | 1.7 |
| NM_001005199 | OR8H1 | 1.7 |
| NM_032145 | FBXO30 | 1.7 |
| NM_004865 | TBPL1 | 1.6 |
| NM_030799 | YIPF5 | 1.6 |
| BC041467 | C17orf67 | 1.6 |
| AL161991 | GPBP1 | 1.6 |
| X97261 | MT1L | 1.6 |
| NM_152829 | TES | 1.6 |
| NM_024685 | BBS10 | 1.6 |
| NM_031435 | THAP2 | 1.6 |
| NM_003299 | HSP90B1 | 1.6 |
| NM_003124 | SPR | 1.6 |
| NM_015608 | C10orf137 | 1.6 |
| NM_006134 | TMEM50B | 1.6 |
| NM_006260 | DNAJC3 | 1.6 |
| AK128731 | ATF2 | 1.6 |
| NM_013275 | ANKRD11 | 1.6 |
| THC2535569 | THC2535569 | 1.6 |
| A_24_P834066 | A_24_P834066 | 1.6 |
| NM_004354 | CCNG2 | 1.6 |
| ENST00000380021 | KIAA0372 | 1.6 |
| NM_000917 | P4HA1 | 1.6 |
| NM_018178 | GOLPH3L | 1.6 |
| NM_003633 | ENC1 | 1.6 |
| NM_012111 | AHSA1 | 1.6 |
| NM_001013398 | IGFBP3 | 1.6 |
| AK094167 | FLJ36848 | 1.6 |
| NM_001079673 | FNDC3A | 1.6 |
| NM_025211 | GKAP1 | 1.6 |
| NM_020727 | ZNF295 | 1.6 |
| NM_182501 | MTERFD2 | 1.6 |
| NM_032385 | C5orf4 | 1.6 |
| AL049246 | SLC25A36 | 1.6 |
| NM_014935 | PLEKHA6 | 1.6 |
| NM_015099 | CAMTA2 | 1.6 |
| NM_014417 | BBC3 | 1.6 |
| NM_014331 | SLC7A11 | 1.6 |
| NM_005868 | BET1 | 1.6 |
| NM_004897 | MINPP1 | 1.6 |
| NM_002357 | MXD1 | 1.6 |
| NM_004064 | CDKN1B | 1.6 |
| NM_032437 | KIAA1799 | 1.6 |
| NM_022173 | TIA1 | 1.6 |
| AK125269 | AK125269 | 1.6 |
| NM_001218 | CAI2 | 1.6 |
| NM_022725 | FANCF | 1.6 |
| NM_020147 | THAP10 | 1.6 |
| BC062636 | RHBDD1 | 1.6 |
| THC2701797 | THC2701797 | 1.5 |
| THC2653489 | THC2653489 | 1.5 |
| AB011115 | LOC643641 | 1.5 |
| THC2515611 | THC2515611 | 1.5 |
| NM_019886 | CHST7 | 1.5 |
| NM_005033 | EXOSC9 | −1.5 |
| NM_001356 | DDX3X | −1.5 |
| ENST00000341865 | FLJ90086 | −1.5 |
| NM_001017430 | RBM3 | −1.5 |
| BC110367 | C15orf39 | −1.5 |
| CR596712 | CR596712 | −1.6 |
| NM_000303 | PMM2 | −1.6 |
| NM_020165 | RAD18 | −1.6 |
| NM_000946 | PRIM1 | −1.6 |
| NM_005441 | CHAF1B | −1.6 |
| BC039449 | MSL-1 | −1.6 |
| NM_001031804 | MAF | −1.6 |
| NM_001238 | CCNE1 | −1.6 |
| NM_145065 | PELI3 | −1.6 |
| A_32_P128399 | A_32_P128399 | −1.6 |
| NM_013282 | UHRF1 | −1.6 |
| NM_014891 | PDAP1 | −1.6 |
| NM_002073 | GNAZ | −1.6 |
| AK055915 | AK055915 | −1.6 |
| NM_023078 | PYCRL | −1.6 |
| NM_017906 | PAKHP1 | −1.6 |
| NM_030928 | CDT1 | −1.6 |
| NM_003586 | DOC2A | −1.6 |
| NM_000179 | MSH6 | −1.6 |
| NM_003720 | DSCR2 | −1.6 |
| NM_005656 | TMPRSS2 | −1.6 |
| NM_014660 | PHF14 | −1.6 |
| NM_004776 | B4GALT5 | −1.6 |
| NM_032932 | RAB11FIP4 | −1.6 |
| NM_001034 | RRM2 | −1.7 |
| NM_033300 | LRP8 | −1.7 |
| NM_016022 | APH1A | −1.7 |
| NM_014550 | CARD10 | −1.7 |
| NM_002916 | RFC4 | −1.7 |
| NM_003211 | TDG | −1.7 |
| NM_003341 | UBE2E1 | −1.7 |
| NM_001031711 | ERGIC1 | −1.7 |
| NM_017742 | ZCCHC2 | −1.7 |
| NM_004762 | PSCD1 | −1.7 |
| NM_004323 | BAG1 | −1.7 |
| NM_031372 | HNRPDL | −1.7 |
| NM_001018051 | POLR3H | −1.7 |
| NM_006805 | HNRPA0 | −1.7 |
| NM_016121 | KCTD3 | −1.7 |
| NM_182924 | MICALL2 | −1.7 |
| AK022609 | FIJ12547 | −1.7 |
| NM_001280 | CIRBP | −1.7 |
| NM_003877 | SOCS2 | −1.7 |
| NM_003468 | FZD5 | −1.7 |
| NM_032336 | GINS4 | −1.8 |
| BC048343 | GPSM1 | −1.8 |
| A_24_P739785 | A_24_P739785 | −1.8 |
| NM_030567 | PRR7 | −1.8 |
| NM_017510 | TMED9 | −1.8 |
| NM_015541 | LR1G1 | −1.8 |
| NM_004091 | E2F2 | −1.8 |
| NM_004117 | FKBP5 | −1.9 |
| NM_007063 | TBC1D8 | −1.9 |
| NM_031922 | REPS1 | −1.9 |
| NM_005573 | LMNB1 | −2.0 |
| NM_194460 | RNF126 | −2.0 |
| NM_006167 | NKX3-1 | −2.0 |
| NM_002437 | MPV17 | −2.0 |
| NM_032292 | GON4L | −2.1 |
| BC045573 | HNRPM | −2.2 |
| NM_033102 | SLC45A3 | −2.2 |
| NM_003325 | HIRA | −2.3 |
| NM_014409 | TAF5L | −2.4 |
| NM_005486 | TOMIL1 | −2.5 |
| NM_006379 | SEMA3C | −3.0 |
| NM_000329 | RPE65 | −4.7 |
| THC2572696 | THC2572696 | −6.3 |
| A_24_P934423 | A_24_P934423 | −7.3 |
| NM_001037171 | ACOT9 | −8.5 |
| M31157 | M31157 | −15.4 |

*All dysregulated genes were selected by Odd ratio ≧10, fold change ≧1.5.

TABLE 4
Representative common dysregulated genes in PC-3 and LNCaP cells*

| | | | PC-3 | LNCaP |
|---|---|---|---|---|
| Systematic | | | fold | fold |
| name | Description | Gene name | change | change |
| NM_005345 | Heat shock 70 kda protein 1A | HSPA1A | 7.7 | 5.8 |
| NM_005527 | Heat shock 70 kda protein 1-like | HSPA1L | 6.0 | 4.7 |
| NM_006644 | Heat shock 105 kda/110 kda | HSPH1 | 2.8 | 3.6 |
| | protein 1 | | | |
| NM_004281 | BCL2-associated athanogene 3 | BAG3 | 2.5 | 3.8 |
| NM_014278 | Heat shock 70 kda protein 4-like | HSPA4L | 2.4 | 2.4 |
| NM_130469 | Jun dimerization protein 2 | JDP2 | 2.1 | 2.2 |
| NM_007034 | Dnaj (Hsp40) homolog, | DNAJB4 | 2.1 | 3.6 |
| | subfamily B, member 4 | | | |
| NM_001114 | Adenylate cyclase 7 | ADCY7 | 2.1 | 1.9 |
| NM_001539 | Dnaj (Hsp40) homolog, | DNAJA1 | 2.1 | 2.1 |
| | subfamily A, member 1 | | | |
| NM_005347 | Heat shock 70 kda protein 5 | HSPA5 | 2.0 | 2.7 |
| NM_001540 | Heat shock 27 kda protein 1 | HSPB1 | 1.9 | 1.7 |
| NM_001040141 | Heat shock protein 90 kda alpha, | HSP90AA2 | 1.9 | 1.7 |
| | class A member 2 | | | |
| NM_004172 | Solute carrier family 1, member 3 | SLC1A3 | 1.8 | 1.7 |
| NM_006145 | Dnaj (Hsp40) homolog, | DNAJB1 | 1.8 | 2.6 |
| | subfamily B, member 1 | | | |
| NM_006010 | Arginine-rich, mutated in early | ARMET | 1.8 | 2.2 |
| | stage tumors | | | |
| NM_012111 | AHA1, activator of heat shock | AHSA1 | 1.8 | 1.6 |
| | 90 kda protein atpase homolog 1 | | | |
| NM_015099 | Calmodulin binding transcription | CAMTA2 | 1.8 | 1.6 |
| | activator 2 | | | |
| NM_001005199 | Olfactory receptor, family 8, | OR8H1 | 1.7 | 1.7 |
| | subfamily H, member 1 | | | |
| NM_052957 | Acidic repeat containing | ACRC | 1.7 | 1.8 |
| NM_182972 | Interferon regulatory factor 2 | IRF2BP2 | 1.7 | 1.7 |
| | binding protein 2 | | | |
| NM_031435 | THAP domain containing, | THAP2 | 1.7 | 1.6 |
| | apoptosis associated protein 2 | | | |
| NM_016303 | WW domain binding protein 5 | WBP5 | 1.6 | 2.1 |
| NM_003124 | Sepiapterin reductase | SPR | 1.6 | 1.6 |
| NM_015513 | Cysteine-rich with EGF-like | CRELD1 | 1.6 | 1.9 |
| | domains 1 | | | |
| NM_000164 | Gastric inhibitory polypeptide | GIPR | 1.6 | 2.0 |
| | receptor | | | |
| NM_032356 | LSM domain containing 1 | LSMD1 | 1.6 | 1.7 |
| NM_002155 | Heat shock 70 kda protein 6 | HSPA6 | 1.6 | 2.0 |
| NM_014660 | PHD finger protein 14 | PHF14 | −1.6 | −1.6 |
| NM_001280 | Cold inducible RNA binding | CIRBP | −1.6 | −1.7 |
| | protein | | | |
| NM_001356 | DEAD (Asp-Glu-Ala-Asp) box | DDX3X | −1.6 | −1.5 |
| | polypeptide 3, X-linked | | | |
| NM_005033 | Exosome component 9 | EXOSC9 | −1.6 | −1.5 |
| NM_001017430 | RNA binding motif protein 3 | RBM3 | −1.7 | −1.5 |
| NM_194460 | Ring finger protein 126 | RNF126 | −1.7 | −2.0 |
| NM_031372 | Heterogeneous nuclear | HNRPDL | −1.9 | −1.7 |
| | ribonucleoprotein D-like | | | |

*All dysregulated genes were selected by Odd ratio ≧10, fold change ≧1.5.

Real-time reverse-transcription PCR (RT-PCR) validation of microarray data: Gene expression changes identified using microarrays were independently verified by real-time RT-PCR. Representative members from both up- and down-regulated genes were selected. As shown in FIGS. 2A-2H, both HSPA1A and HSPA6 were found to be up-regulated upon heat treatment at 41° C. while both the cold shock proteins RBM3 and CIRBP were down regulated at the same temperature in both the prostate cell lines.

Immunoblotting confirms differential expression a protein level: Having confirmed the gene expression changes at the RNA level, the protein levels were evaluated. Immunoblotting analysis of total cell lysates from both cell lines demonstrated that Hspa1a increased in expression within 4 h of heat administration (FIGS. 3A-3B). However, unlike the RNA levels, a sustained increased expression of the Hspa1a protein was observed over the 24 h study period. On the other hand, both RBM3 and CIRBP proteins are significantly down regulated during the same period (FIGS. 3A-3B). Considered together, the data clearly demonstrate that mild heat treatment results in the up-regulation of a family of heat shock proteins and down-regulation of members of the cold-shock proteins both at the mRNA as well as the protein level.

siRNA-mediated knockdown of cold shock proteins as a potential “heat mimic”: Cold shock proteins as a class include RNA-binding proteins and have been reported to be up-regulated in response to cold and other forms of stress analogous to the HSPs (11). Despite this, it is not readily apparent why these genes are down-regulated in response to heat-induced stress. In order to gain additional insight, the expression of the cold shock transcripts was knocked down to “mimic” the heat-shock effect. Mimicking the effect of heat-shock was found to enhance the susceptibility of these cells to therapeutic treatment in the absence of any heat. The levels of the RBM3 and CIRBP transcripts were estimated in siRNA-treated cells to determine the knockdown effects,

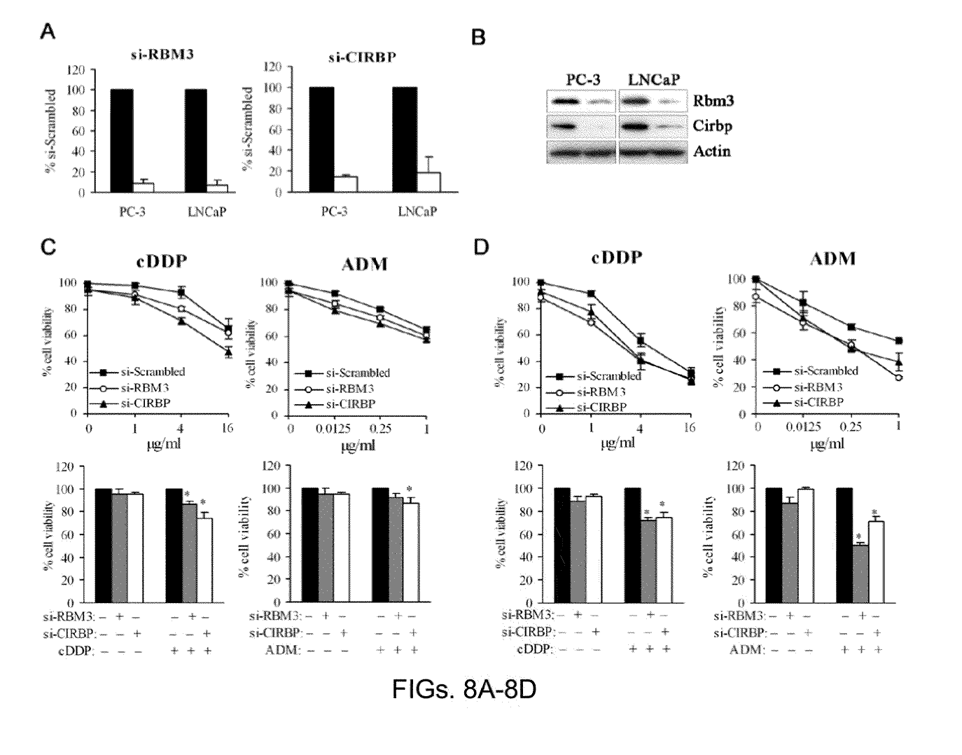

PC-3 and LNCaP cells were transfected with 100 nM RBM3 or CIRBP SMARTpool siRNA. Forty-eight hours after transfection, total RNA was isolated and mRNA levels of RBM3 and CIRBP were detected by real-time RT-PCR (FIG. 8A), or cells were lysed and subjected to Western blot to detect RBM3 and CIRBP levels (FIG. 8B). Twenty-four hours after siRNA transfection, PC-3 cells (FIG. 8C) and LNCaP cells (FIG. 8D) were treated with increasing doses of cisplatin (cDDP) or adriamycin (ADM) for 4 h and then moved back to normal medium for another 72 h. Cell viability inhibition was determined using the WST-1 assay. In the lower panel of (FIG. 8C) and (FIG. 8D), cell viability was normalized to the scrambled siRNA-knock down cells, which was treated with the same dose of drug (cDDP, 4 μg/ml; ADM, 1 μg/ml). Data are represented as mean±SD. *, P<0.05 compared to scrambled siRNA-treated control.

As shown in FIG. 8A, siRNAs specific to either RBM3 or CIRBP decreased the levels of their respective mRNAs by 85-95% (open bars) compared to a non-specific (scrambled) siRNA (solid bars) used at the same concentration. The RBM3 and CIRBP proteins were also similarly affected by the knockdown, and as evident from FIG. 8B the proteins were reduced to almost undetectable levels. Having confirmed that the siRNAs abolished the expression of the RBM3 and CIRBP proteins. CIRBP or RBM3 expression was knocked down and the cells subsequently treated with adriamycin (ADM) or cisplatin (cDDP). As shown in FIGS. 8C and 8D, knocking down these genes resulted in a marked reduction of cell survival by ˜25-50% in PC-3 and LNCaP cells, respectively compared to the drug treatment alone. However, unlike the case of CIRBP where the knock-down had a similar effect on synergizing cDDP and ADM cytotoxicity in both cell lines, RBM3 knock-down was more effective in LNCaP cells than in PC-3 cells suggesting that RBM3 function may be cell type-dependent.

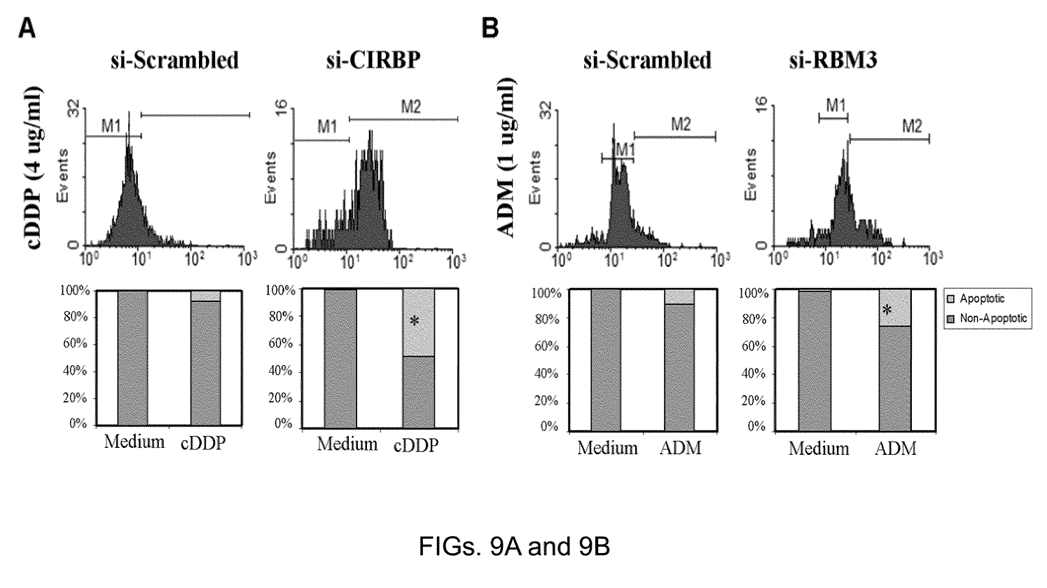

The enhancement of RBM3 and CIRBP knockdown on chemosensitivity was also confirmed by detecting apoptosis using the Terminal Transferase dUTP Nick End Labeling (TUNEL) assay. Apoptosis induced by chemotherapy was enhanced by RBM3 or CIRBP knock-down. PC-3 cells (FIG. 9A) and LNCaP cells (FIG. 9B) were transfected with 100 mM RBM3 or CIRBP SMARTpool siRNA. Forty-eight hours after transfection, cells were treated with 4 μg/ml cDDP or 1 μg/ml ADM for 4 h, and then moved back to normal medium for another 16 h. Cells were collected by centrifugation and subjected to Terminal Transferase dUTP Nick End Labeling (TUNEL) assay. Apoptolic and non-apoptotic cells population was divided using flow cytometry. *, P<0.01 compared to scrambled siRNA-treated control. (FIGS. 9A and 9B).

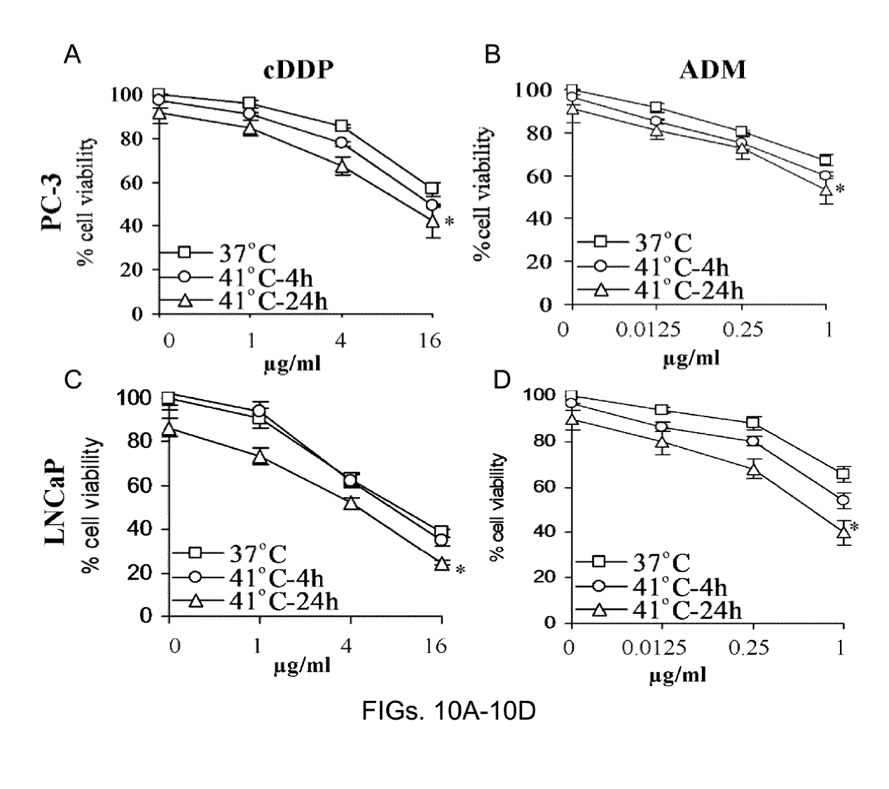

Heating the cells at 41° C. combined with drug treatment also resulted in a similar decrease in cell survival, as shown in FIGS. 10A-10D. PC-3 cells (FIGS. 10A and 10B) and LNCaP cells (FIGS. 10C and 10D) were treated at 41° C. for 4 h, 24 h, or maintained at 37° C. as a control. cDDP or ADM were added during the heat treatment and washed out 4 h later. Following heat and drug treatment, cells were maintained at 37° C. under routine conditions for another 72 h. Cell viability was determined using the WST-1 assay. All experiments were repeated at a minimum of two times. Data are represented as mean±SD. *, P<0.05 compared to 37° C. control. The results in FIGS. 10A-10D highlight the heat mimicking effects of the siRNAs, and the synergy when combined with chemotherapy.

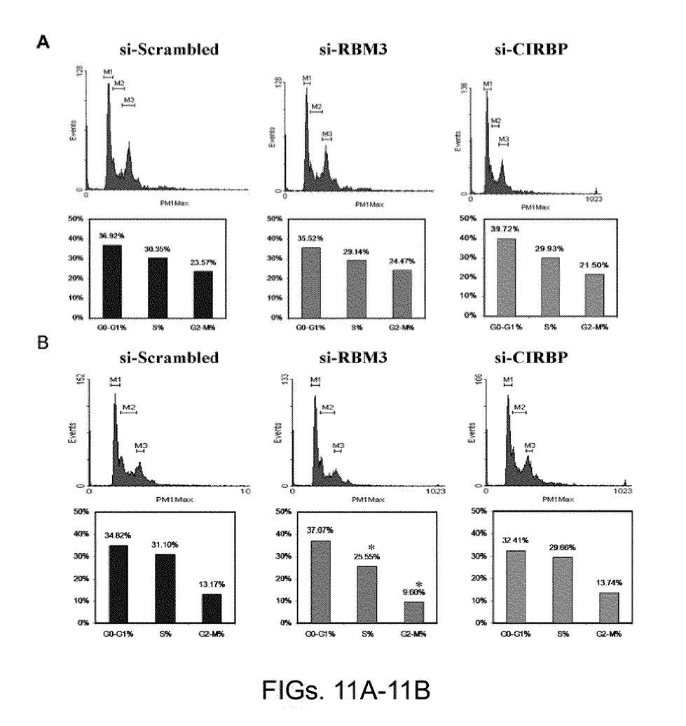

RBM3 and CIRBP act via different mechanisms: To address the mechanism by which RBM3 and CIRBP might modulate the heat mimic, potential cellular pathways were explored. A TUNEL assay revealed that knocking-down RBM3 or CIRBP alone (in the absence of any drug) failed to induce apoptosis in either prostate cancer cell line. In contrast, RBM3 down-regulation induced a cell cycle arrest before the S phase and G2-M phase in LNCaP but not PC-3 cells, PC-3 cells (FIG. 11A) and LNCaP cells (FIG. 11B) were transfected with 100 nM RBM3 or CIRBP SMARTpool siRNA. Seventy-two hours after transfection, cells were collected by centrifugation, stained with propidium iodide (PI) and subjected to DNA content analysis using the flow cytometry. *, P<0.05 compared to scrambled siRNA-treated control.

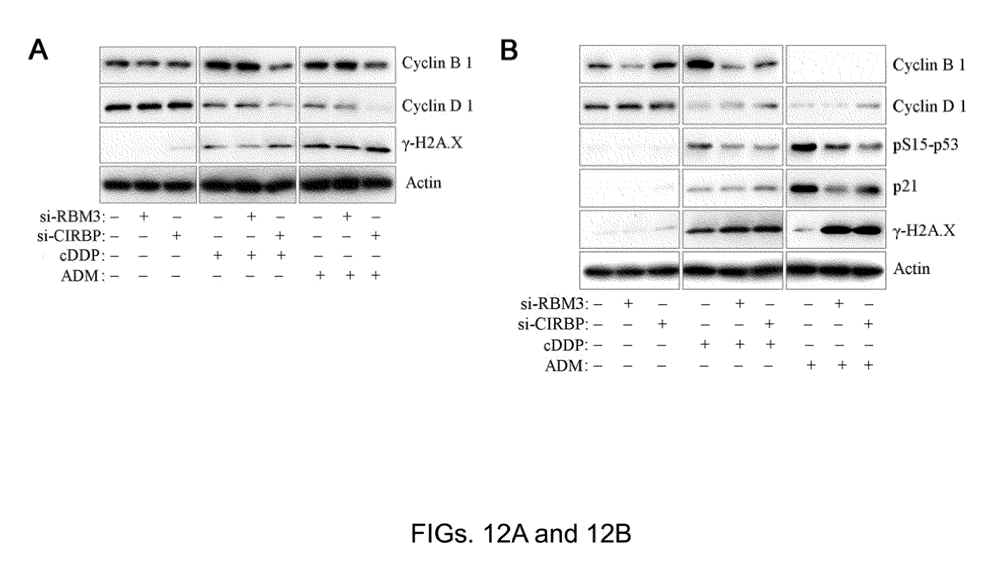

Consistent with the cell cycle analysis, knocking down RBM3 reduced cyclin B1 protein levels in LNCaP but not in PC-3 cells (see FIGS. 12B and 12A, respectively). Prostate cancer cells PC-3 (FIG. 12A) and LNCaP (FIG. 12B) were transfected with 100 nM RBM3 or CIRBP SMARTpool siRNA. Forty-eight hours after transfection, cells were treated with 4 μg/ml cDDP or 1 μg/ml ADM for 4 h, and then moved back to normal medium for another 16 h. Cell lysates were subjected to Western blotting to detect Cyclin B1, Cyclin D1, phosph-histone H2A.X (Ser139) (γ-H2A.X), phosph-p53 (Ser15), and p21. Actin was used as a control for protein loading. Cyan B1 levels were not increased in response to drug treatment in RBM3 knocked-down LNCaP cells as it was in the scrambled siRNA-treated control cells (FIG. 12B) corroborating the fact that RBM3 down-regulation in conjunction with drug treatment is more effective in LNCaP cells than in PC-3 cells (FIGS. 8C and 8D).

Alternatively, knocking down CIRBP in either PC-3 or LNCaP cells did not have any significant impact on either cell cycle or cyclin B1 and D1 levels (FIGS. 11A and 11B). However, the levels of cyclin B1 and/or cyclin D1 in CIRBP knocked-down in either cell line were significantly lower than scrambled siRNA-treated control after treating cells with drug (FIGS. 12A and 12B). Together with the fact that CIRBP knock-down similarly synergized chemosensitivity in both PC-3 and LNCaP cells, these results suggest that CIRBP acts via a mechanism distinct from that of RBM3.

RBM3 and CIRBP appear to he involved in DNA damage repair: It is generally accepted that DNA damage and subsequent cell death may be the primary cytotoxic mechanism of ADM and other DNA-binding antitumor drugs. However, since knocking down RBM3 or CIRBP did not induce apoptosis or only partly impeded the cell cycle, it was determined if the knock-down promotes DNA damage repair rendering the cells more susceptible to the effects of chemotherapy. Since phosphorylation of H2A.X at serine 139 correlates well with DNA damage (12), we determined the phosph-histone H2A.X (γ-H2A.X) content to evaluate the impact of the knock-down on DNA damage and repair response. As shown in FIG. 12B, both RBM3 and CIRBP down-regulation significantly enhanced DNA damage triggered by drugs in LNCaP cells as judged by the increase in accumulation of γ-R2A.X. Again, only CIRBP but not RBM3 knocked-down increased cDDP and ADM induced DNA damage in PC-3 cells, further suggesting that RBM3 function is different between two cell lines (FIG. 12A). Notably, even without any chemical stress, knocking down of CIRBP still can induce a slight but identifiable increase of DNA damage marker.

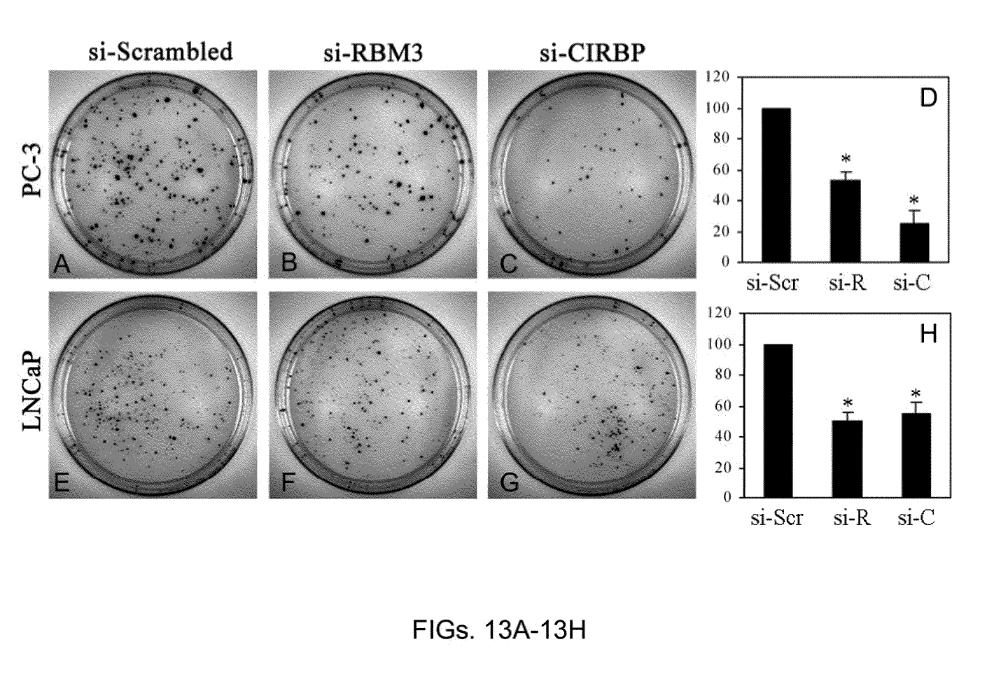

Finally, since p53-p21 proteins play a key role in protecting the cell from DNA damage-induced cell death (13), their expression in LNCaP cells that expresses wild type p53 was determined. As expected, knocking down both RBM3 and CIRBP significantly inhibited the activation of p53. In contrast, p53 was activated by cDDP and ADM treatment in scrambled siRNA controls. The up-regulation of p21 induced by ADM was also impeded in LNCaP cells. Supporting these results, colony formation that mainly relies on the intactness of the DNA damage response pathway, was significantly inhibited in a clonogenic assay after knocking-down these two genes in both cell lines (FIGS. 13A-13H). PC-3 and LNCaP cells were transfected with 100 nM RBM3 or CIRBP SMARTpool siRNA. Forty-eight hours after transfection, cells were re-plated in 10-cm dish with 200 cells per dish, and colonies were counted after 2-week growing. All experiments were repeated at a minimum of two times. *, P<0.01 compared to scrambled siRNA-treated control. These results suggest that RBM3 and CIRBP may be involved in the DNA damage response process. Consequently, down-regulating them, enhances the effects of chemotherapy.

### Discussion

The benefit of combining heat treatment along with other therapeutic approaches is an area of significant promise in the treatment of cancer. However, the mechanisms leading to favorable clinical results of heat therapy have not been fully understood prior to the present work. By interrogating global gene expression profiles and the nuclear matrix-associated proteome, some of the key heat-induced alterations in the tumor microenvironment using cell line models of cancer have now been identified by the present inventors.

As expected, an overwhelming up-regulation of HSPs and genes related to the cancer cell's innate heat shock response was observed. However, of the dozens of stress-induced HSPs in a cancer cell, only a few have been found to have critical cytoprotective roles in cancer such as HSP27 and HSP70 (14). Both proteins are powerful chaperones, inhibit key effectors of the apoptotic machinery, and participate in the proteasome-mediated degradation of proteins under stress conditions, thereby contributing to the so called “protein triage.” In cancer cells, both HSP27 and HSP70 appear to participate in oncogenesis and in resistance to chemotherapy. Thus, in animal models, while their over-expression increases tumor growth and metastatic potential, inhibiting expression frequently reduces the size of the tumors (15-17) and even can cause their complete involution (for HSP70). Therefore, the inhibition of HSP70 and HSP27 has become a novel strategy for cancer therapy (18).

HSP27 and HSP70 were up-regulated in both cell lines studied here. However, while HSP70 was detected both in gene expression and nuclear matrix proteomic analyses, HSP27 over-expression was only observed in the gene expression profiling studies. Interestingly, it has been reported by other investigators that Hsp70 binds to the nuclear matrix (8) and thus, is detected as an integral component of the nuclear matrix by two-dimensional electrophoresis. On the other band, Hsp27 does not appear to bind to the nuclear matrix directly. In fact, Hap27 interacts indirectly by associating with Saf-b, a constituent of the nuclear matrix (19) and thus, may not withstand the harsh extraction procedures underlying nuclear matrix preparations.

Notably, all organisms from prokaryotes to plants and higher eukaryotes respond to cold shock in a comparatively similar manner. Cold shock invokes the rapid over-expression of as small group of proteins called cold-shock proteins, and a coordinated cellular response involving modulation of transcription, translation, metabolism, the cell cycle and the cell cytoskeleton. In mammalian cells however, to date, only two cold-shock proteins have been described in detail in, CIRBP (Accession No. BAA11212) and RBM3 (Accession No. NM006743) (20). There is now growing evidence that in addition to their role in cold stress response, the cold shock proteins also play critical roles in cancer cell survival and growth. For example, CIRBP is induced by stresses such as UV light and hypoxia (21). Indeed, using the RKO colorectal carcinoma cells, Yang & Carrier (22) observed that cells expressing reduced levels of CIRBP are more sensitive to UV light than those over-expressing CIRBP.

The extraordinary success in treating Lance Armstrong with distant metastasis of testicular cancer is suggested to be due, at least in part, to its susceptibility to the increased body temperature experienced by the metastatic cells (3). Interestingly, when the mouse testis was exposed to heat stress by experimental cryptorchidism or immersion of the lower abdomen in warm (42° C.) water, the expression of CIRBP was decreased in the testis within 6 hours after either treatment (23). The authors also observed that in human testis with varicocele when analyzed immunohistochemically, germ cells expressed less CIRBP protein than those in the testis without varicocele (23). Together, these observations support the argument that the down-regulation of CIRBP in response to heat may he involved in the tumor cell's susceptibility to therapy.

RBM3 is one of the first proteins synthesized in response to cold shock (24). Although the exact biological function of RBM3 is not fully understood, members of the RBM protein family contain the primary structural motif most commonly referred to as the RNA-recognition motif (RRM) and are thought to function as RNA binding apoptosis regulators (11). RRMs are also known as RNA-binding domain or ribonucleoprotein domain (RNP), Examples of RRMs include RNP-1 (AVFSLSQPEQVKIAVNTS KYASES) (SEQ ID NO: 19) and RNP-2 (VLHVTFPKEWKTSDLYQLFSAFGNI) (SEQ ID NO: 20). Sequences suitable for down-regulation of RRMs including RNP-1 and RNP-2, for example using antisense or siRNA, are well known or can be designed, and are readily developed and prepared by those of ordinary skill in the art (see e.g., Frese et al., Journal of Thoracic and Cardiovascular Surgery 126: 748-754 (2003); Hosaka et al., Cancer Science 97:623-632 (2006); Friedman, Nature Biotechnology 26:399-400 (2008); Hagiwara et al, Respiratory Research 8:37 (2007); Hadaschik et al., British Journal of Urology 102:610-616 (2008); and McGarry and Lindquist, Proceedings of the National Academy of Sciences 83:399-103 (1986), the disclosures of each of which are incorporated by reference herein in their entireties.

RBM3 in particular, appears to be a novel proto-oncogene that induces transformation when over-expressed and is essential for cells to progress through mitosis (25). While over-expression increases cell proliferation and development of compact multicellular spheroids in soft agar, down-regulating RBM3 in HCT116 colon cancer cells with specific siRNA decreases cell growth in culture and tumor xenografts (25). Down-regulation also increases caspase-mediated apoptosis coupled with nuclear cyclin B1, and phosphorylated Cdc25c, Chk1 and Chk2 kinases, implying that under conditions of RBM3 down-regulation, cells undergo mitotic catastrophe (25). Like in colon cancer, RBM3 is also up-regulated in prostate cancer. However, unlike in colon cancer wherein there is a stage-dependent increase (25), RBM3 is apparently down-regulated in late-stage prostate cancer. Further, in contrast to colon cancer wherein its down-regulation leads to mitotic catastrophe, the present data indicate that in androgen sensitive, but not in androgen independent prostate cancer cells, down-regulation of RBM3 leads to a cell cycle arrest and an enhancement of DNA damage induced by drug suggesting tumor-specific differences and warrants further investigation.

The results presented herein on the differential effects of knocking down RBM3 on the therapeutic efficacy of cytotoxic drugs in two prostate cancer cell lines may be predicated on the p53 status of the cell models utilized. In response to DNA damage, induction of p53 protein either causes the cells to arrest in different phases of the cell cycle, or if DNA damage is excessive, p53 leads the cells through apoptosis by regulating the bcl-2 family of genes (26, 27). Of particular note is the fact that LNCaP cells possess wild-type p53 while PC-3 has mutant p53 that cannot be functionally activated following DNA damage (28). It has been shown that the susceptibility of these prostate cancer cells to cDDP and its analogs appears to be linked to the p53 status (29). Indeed, in the present study it was observed that knocking down RBM3 impedes p53 activation and the subsequent p21 expression, both of which previously have been shown to render DNA damage repair (13), when LNCaP cells was subjected to a cytotoxic stress. Considered together, it appears that RBM3 may be involved in p53-linked DNA damage repair. In contrast, while CIRBP knock-down similarly altered p53-p21 proteins and increased γ-H2A.X expression under the chemical stress in LNCaP cells, it is also able to enhance DNA damage and cytotoxic killing in PC-3 cells in which p53 regulation is deficient. This p53-independent pathway appears to impair cyclin B1 increase and enhance cyclin D1 decrease after drug treatment. Together, the present data support the notion that RBM3 and CIRBP appear to involve different cell death resistant mechanisms in different types of cancer cells.

In summary, the down-regulation of the cold shock proteins, and the response to heat treatment, enhance the sensitivity of heat-treated cells to subsequent therapeutic modalities, albeit by different mechanisms. The present invention also provides a further combination of modalities, such as a double knockdown of the HSPs and cold shock proteins for example, coupled with other forms of chemo- or radiation therapy, to further enhance the synergism and therapeutic effectiveness.

### REFERENCES

1. Shamovsky I & Nudler E (2008) New insights into the mechanism of heat shock response activation. Cell Mol Life Sci 65:855-861.
2. Dewhirst M W. Vujaskovic Z., Jones E, &. Thrall D (2005) Re-setting the biologic rationale for thermal therapy. Int. J Hyperthermia 21:779-790.
3. Coffey D S, Getzenberg R H, & DeWeese T L (2006) Hyperthermic biology and cancer therapies: a hypothesis for the “Lance Armstrong effect”. JAMA 296:445-448.
4. Berezney R, Mortillaro M J, Ma H, Wei X, & Samarabandu J (1995) The nuclear matrix: a structural milieu for genomic function. Int Rev Cytol 162A:1-65.
5. Pederson T (2000) Half a century of “the nuclear matrix”. Mol Biol Cell 11:799-805.
6. Getzenberg R H, et al. (1996) Bladder cancer-associated nuclear matrix proteins. Cancer Res 56:1690-1694.
7. Roti Roti J L, et al. (1998) Nuclear matrix as a target for hyperthermic killing of cancer cells. Cell Stress Chaperones 3:245-255.
8. Lepock J R, Frey H E, Heynen M L. Senisterra G A, & Warters R L (2001) The nuclear matrix is a thermolabile cellular structure. Cell Stress Chaperones 6:136-147.
9. Roti Roti J L (2008) Cellular responses to hyperthermia (40-46 degrees C.): cell killing and molecular events. Int J Hyperthermia 24:3-15.
10. Myerson R J, Roti Roti J L, Moros E G, Straube W L, & Xu M (2004) Modeling heat-induced radiosensitization: clinical implications. Int J Hyperthermia 20:201-212.
11. Sutherland L C, Rintala-Maki N D, White R D, & Morin C D (2005) RNA binding motif (RBM) proteins: a novel family of apoptosis modulators? J Cell Biochem 94:5-24.
12. Plesca D, Mazumder S, &. Almasan A (2008) DNA damage response and apoptosis. Methods Enzymol 446:107-122.
13. Garner E & Raj K (2008) Protective mechanisms of p53-p21-pRb proteins against DNA damage-induced cell death. Cell Cycle 7:277-282.
14. Garrido C, et al. (2006) Heat shock proteins 27 and 70: anti-apoptotic proteins with tumorigenic properties. Cell Cycle 5:2592-2601.
15. Zhao Z G, Ma Q Z, & Xu C X (2004) Abrogation of heat-shock protein (HSP)70 expression induced cell growth inhibition and apoptosis in human androgen-independent prostate cancer cell line PC-3m. Asian J Androl 6:319-324,
16. Rocchi P. et al. (2006) Small interference RNA targeting heat-shock protein 27 inhibits the growth of prostatic cell lines and induces apoptosis via caspase-3 activation in vitro. BJU Int 98:1082-1089.
17. Kamada M. et al. (2007) Hsp27 knockdown using nucleotide-based therapies inhibit tumor growth and enhance chemotherapy in human bladder cancer cells. Mol Cancer Ther 6:299-308.
18. Didelot C. et al. (2007) Anti-cancer therapeutic approaches based on intracellular and extracellular heat shock proteins. Curr Med Chem 14:2839-2847.
19. Oesterreich S. et al. (1997) Novel nuclear matrix protein HET binds to and influences activity of the HSP27 promoter in human breast cancer cells. J Cell Biochem 67:275-286.
20. Al-Fageeh M B & Smales C M (2006) Control and regulation of the cellular responses to cold shock: the responses in yeast and mammalian systems. Biochem J 397:247-259.
21. De Leeuw F, et al. (2007) The cold-inducible RNA-binding protein migrates from the nucleus to cytoplasmic stress granules by a methylation-dependent mechanism and acts as a translational repressor. Exp Cell Res 313:4130-4144.
22. Yang C & Carrier F (2001) The UV-inducible RNA-binding protein A18 (A18 hnRNP) plays a protective role in the genotoxic stress response. J Biol Chem 276:47277-47284.
23. Nishiyama H, et al. (1998) Decreased expression of cold-inducible RNA-binding protein (CIRP) in male germ cells at elevated temperature. Am J Pathol 152:289-296.
24. Danno S, et al. (1997) Increased transcript level of RBM3, a member of the glycine-rich RNA-binding protein family, in human cells in response to cold stress. Biochem Biophys Res Commun 236:804-807,
25. Sureban S M, et al. (2008) Translation regulatory factor RBM3 is a proto-oncogene that prevents mitotic catastrophe. Oncogene 27:4544-4556.
26. Weinberg R A (1991) Tumor suppressor genes. Science 254:1138-1146.
27. Lane D P (1992) Cancer. p 53, guardian of the genome. Nature 358:15-16.
28. O'Connor P M, et al. (1997) Characterization of the p53 tumor suppressor pathway in cell lines of the National Cancer Institute anticancer drug screen and correlations with the growth-inhibitory potency of 123 anticancer agents. Cancer Res 57:4285-4300.
29. Mujoo K, Watanabe M, Khokbar A R, & Siddik Z H (2005) Increased sensitivity of a metastatic model of prostate cancer to a novel tetravalent platinum analog. Prostate 62:91-100,

Exemplary embodiments of the present invention have been presented. The invention is not limited to these examples. These examples are presented herein for purposes of illustration, and not limitation. Alternatives (including equivalents, extensions, variations, deviations, etc., of those described herein) will be apparent to persons skilled in the relevant art(s) based on the teachings contained herein. Such alternatives fall within the scope and spirit of the invention.

All publications, patents and patent applications mentioned in this specification are herein incorporated by reference to the same extent as if each individual publication, patent or patent application was specifically and individually indicated to be incorporated by reference.

What is claimed is:

1. A method of treating a patient suffering from a diseased tissue, comprising:
administering to the diseased tissue of the patient, one or more nucleic acid molecules that down-regulate one or more cold shock proteins in the diseased tissue,
wherein the susceptibility of the diseased tissue to an additional therapy is enhanced relative to diseased tissue which has not been administered one or more nucleic acids.

2. The method of claim 1, wherein the nucleic acid molecules are siRNA.

3. The method of claim 2, wherein the siRNA down-regulate the cold shock proteins RBM3 and/or CIRBP.

4. The method of claim 1, wherein the diseased tissue is a cancerous tissue.

5. The method of claim 4, wherein the cancerous tissue is a cancerous tissue of the heart, lung, breast, prostate, bladder, pancreas, brain, stomach, kidney, testes, lymph nodes, skin, bone or bone marrow.

6. The method of claim 1, further comprising administering to the patient an additional therapy selected from the group consisting of a chemotherapeutic agent, radiation therapy, immunotherapy, radio-immunotherapy, gene therapy and gene silencing therapy.

7. The method of claim 6, wherein the chemotherapeutic agent comprises an agent selected from the group consisting of methotrexate, adriamycin, epirubicin, daunorubicin, doxorubicin, amphotericin B, vincristine, vinblastine, etoposide, ellipticine, camptothecin, paclitaxel, docetaxol, cisplatin, prednisone, methyl-prednisone, and navalbene.

8. The method of claim 1, further comprising administering one or more additional nucleic acid molecules to the diseased tissue that down-regulate one or more heat shock proteins in the diseased tissue.

9. The method of claim 8, wherein the additional nucleic acid molecules are siRNA,

10. The method of claim 1, wherein the patient is a mammal.

11. The method of claim 10, wherein the patient is a human.

12. A method of treating a patient suffering from a diseased tissue, comprising:
environmentally stressing the diseased tissue of the patient for a period of greater than about 10 minutes;
administering to the diseased tissue of the patient one or more nucleic acid molecules that down-regulate one or more cold shock proteins in the diseased tissue; and
administering an additional therapy to the patient selected from the group consisting of a chemotherapeutic agent, radiation therapy, immunotherapy, radio-immunotherapy, gene therapy and gene silencing therapy.
wherein the susceptibility of the diseased tissue to the additional therapy is enhanced relative to diseased tissue which has not been environmentally stressed and administered one or more nucleic acids

13. The method of claim 12, wherein the environmental stressing comprises locally heating the diseased tissue, so as to raise the temperature of the diseased tissue to about 39° C. to about 41° C. for a period of greater than about 10 minutes

14. The method of claim 12, wherein the diseased tissue is a cancerous tissue.

15. The method of claim 14, wherein the cancerous tissue is a cancerous tissue of the heart, lung, breast, prostate, bladder, pancreas, brain, stomach, kidney, testes, lymph nodes, skin, bone or bone marrow.

16. The method of claim 13, wherein the temperature is raised for a period of about 30 minutes to about 24 hours.

17. The method of claim 16, wherein the temperature is raised for a period of about 4 hours to about 8 hours.

18. The method of claim 13, wherein the temperature is raised to about 39° C.

19. The method of claim 13, wherein the temperature is raised to about 40° C.

20. The method of claim 13, wherein the temperature is raised to about 41° C.

21. The method of claim 13, wherein the local heating comprises application of non-ionizing electromagnetic radiation, ionizing radiation or sound energy.

22. The method of claim 13, wherein the local heating comprises administering a magnetic material to the patient and applying an alternating magnetic field so as to inductively beat the magnetic material.

23. The method of claim 12, wherein the chemotherapeutic agent comprises an agent selected from the group consisting of methotrexate, adriamycin, epirubicin, daunorubicin, doxorubicin, amphotericin B, vincristine, vinblastine, etoposide, ellipticine, camptothecin, paclitaxel, docetaxol, cisplatin, prednisone, methyl-prednisone, and navalbene.

24. The method of claim 12, further comprising administering one or more additional nucleic acid molecules to the tissue that down-regulates one or more heat shock proteins in the tissue.

25. The method of claim 24, wherein the additional nucleic acid molecules are siRNA.

26. The method of claim 12, wherein the nucleic acid molecules are siRNA.

27. The method of claim 26, wherein the siRNA down-regulate the cold shock proteins RBM3 and/or CIRBP.

28. The method of claim 12, wherein the patient is a mammal.

29. The method of claim 28, wherein the patient is a human.

30. A method of identifying a tissue in which cold shock proteins are down-regulated in response to environmental stressing of the tissue, comprising:
environmentally stressing the tissue for a period of greater than about 10 minutes;
assaying the tissue for expression of one or more cold shock proteins; and
comparing the expression of the cold shock proteins to the expression of cold shock proteins in a sample of the tissue that has not been environmentally stressed, wherein a decrease in the expression in the environmentally stressed sample relative to the expression in the non-environmentally stressed sample identifies the environmentally stressed sample as a tissue in which cold shock proteins are down-regulated in response to the environmental stressing,

31. The method of claim 30, wherein the environmental stressing comprises heating the tissue, so as to raise the temperature of the tissue to about 39° C. to about 41° C. for a period of greater than about 10 minutes.

32. The method of claim 31, wherein the temperature is raised for a period of about 30 minutes to about 24 hours.

33. The method of claim 32, wherein the temperature is raised for a period of about 4 hours to about 8 hours.

34. The method of claim 31, wherein the temperature is raised to about 39° C.

35. The method of claim 31, wherein the temperature is raised to about 40° C.

36. The method of claim 31, wherein the temperature is raised to about 41° C.

37. The method of claim 30, wherein the assaying for expression comprises analysis of RNA from the tissue.

38. The method of claim 30, wherein the assaying for expression comprises analysis of protein from the tissue.

39. The method of claim 30, wherein the tissue is a mammalian tissue.

40. The method of claim 39, wherein the tissue is a human tissue.

41. A method of treating a patient suffering from a diseased tissue, comprising:
identifying a diseased tissue of the patient in which cold shock proteins are down-regulated in response to environmental stressing of the diseased tissue; and
environmentally stressing the diseased tissue of the patient for a period of greater than about 10 minutes.
wherein the susceptibility of the diseased tissue to an additional therapy is enhanced relative to diseased tissue that has not been environmentally stressed.

42. The method of claim 41, wherein the environmental stressing comprises locally heating the diseased tissue, so as to raise the temperature of the tissue to about 39° C. to about 41° C. for a period of greater than about 10 minutes

43. The method of claim 41, wherein the diseased tissue is a cancerous tissue.

44. The method of claim 41, wherein the cancerous tissue is a cancerous tissue of the heart, lung, breast, prostate, bladder, pancreas, brain, stomach, kidney, testes, lymph nodes, skin, bone or bone marrow.

45. The method of claim 42, wherein the temperature is raised for a period of about 30 minutes to about 24 hours.

46. The method of claim 45, wherein the temperature is raised for a period of about 4 hours to about 8 hours.

47. The method of claim 42, wherein the temperature is raised to about 39° C.

48. The method of claim 42, wherein the temperature is raised to about 40° C.

49. The method of claim 42, wherein the temperature is raised to about 41° C.

50. The method of claim 42, wherein the local heating comprises application of non ionizing electromagnetic radiation, ionizing radiation or sound energy.

51. The method of claim 42, wherein the local heating comprises administering a magnetic material to the patient and applying an alternating magnetic field so as to inductively heat the magnetic material.

52. The method of claim 41, further comprising administering to the patient one or more additional therapies selected from the group consisting of a chemotherapeutic agent. radiation therapy, immunotherapy, radio-immunotherapy, gene therapy and gene silencing therapy.

53. The method of claim 52, wherein the chemotherapeutic agent comprises an agent selected from the group consisting of methotrexate, adriamycin, epirubicin, daunorubicin, doxorubicin, amphotericin B, vincristine, vinblastine, etoposide, ellipticine, camptothecin, paclitaxel, docetaxol, cisplatin, prednisone, methyl-prednisone, and navalbene.

54. The method of claim 41, further comprising administering one or more nucleic acid molecules to the tissue that down-regulate one or more heat shock proteins in the tissue.

55. The method of claim 54, wherein the nucleic acid molecules are siRNA.

56. The method of claim 41, further comprising administering one or more nucleic acid molecules that down-regulate one or more cold shock proteins to the tissue.

57. The method of claim 56, wherein the nucleic acid molecules are siRNA.

58. The method of claim 57, wherein the siRNA down-regulate the cold shock proteins RBM3 and/or CIRBP.

59. The method of claim 41, wherein the patient is a mammal.

60. The method of claim 59, wherein the patient is a human.
